SEQUENCE LISTING (49 sequences)

SEQ ID NO: 1 - protein, 138 aa, synthetic construct
PYIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 2 - protein, 173 aa, synthetic construct
EDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 3 - protein, 9 aa, synthetic construct
ALYDKTKRI

SEQ ID NO: 4 - DNA, 789 bp, synthetic construct
  source 1..789: mol_type = other DNA
atggccttaccagtgaccgccttgctcctgccgctggccttgctgctccacgccgccaggccggctcaagttcagctggtgcagagcggagcggaggtgaagaagccagggtcttccgttaaggtctcttgtaaggcctcaggcggaactttctcatcttacgctatttcctgggtaaggcaagcaccaggacagggattagaatggatgggggggattatccccattttcggtaccgcgaactacgcccaaaaatttcaaggtcgggtaaccatcaccgccgatgaatcgacatctaccgcctacatggaactgagctctcttcggagcgaggacaccgcagtctattactgcgcgagagacgggtatagtggtagctactactattattacggcatggacgtttggggccaggggaccttagtgacggtgagcagcggaggaggcggcgggtcccagtctgctctgacccaacctgccagcgtctccggcagtccaggacaaagtatcaccatcagctgcactgggacttccagcgatgtggggggctacaattacgtaagttggtaccagcaacaccctgggaaggcccccaagctgatgatttatgatgtctcatacagaccgagcggagtgagccaccgttttagtggctccaagtccggaaacaccgcttcactaacaatcagtggcttacaagcagaggacgaagccgattattactgctcatcttacaccagttccagtacgcttgtgtttggtactggaacaaagttgactgtgctgggc

SEQ ID NO: 5 - protein, 262 aa, synthetic construct
MALPVTALLLPLALLLHAARPAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGYSGSYYYYYGMDVWGQGTLVTVSSGGGGGSQSALTQPASVSGSPGQSITISCTGTSSDVGGYNYVSWYQQHPGKAPKLMIYDVSYRPSGVSHRFSGSKSGNTASLTISGLQAEDEADYYCSSYTSSSTLVFGTGTKLTVL

SEQ ID NO: 6 - DNA, 801 bp, synthetic construct
  source 1..801: mol_type = other DNA
atggccttaccagtgaccgccttgctcctgccgctggccttgctgctccacgccgccaggccggcccaagtccagctggttgaaagcgggggaggagtggttcagcccggaaggagtttgcggttgagttgtgcagcatcaggattcacattttcttcctatgcaatgtcctgggtgcgtcaggctcccggaaagggactggaatgggttagcgctatatctggcagcggagactctacatattatgctgattcagtcaaaggccgtttcaccatcagcagagacaacagtaagaataccctctaccttcagatgaactcgctacgtgctgaagacaccgccgtttactactgtgctaaagatctcgactcctcaagtccggacgatgcttttgacatctggggacaaggcaccactgtcaccgtcagcagcggaggcgccgcgctggccgaagtggcagccgcggtggctgaccctatcgttctgactcagtctcctggaaagctgagcctctccccaggggagcgggccactttgtcctgtggcgcgtcgcagagcgtatcatccaattacctggcatggtaccagcagaaacctggcctggctcctcggcttttgatttatgatgctagtattagggctactggcgtgcctgatcggttctcgggatcagggagcgcgactgacttcactttgacgatcagccggctggaccctgaggattttgcggtgtactattgccaccagtattcatctgcccccatgacttttggacagggcactaagctggagattaagagg

SEQ ID NO: 7 - protein, 267 aa, synthetic construct
MALPVTALLLPLALLLHAARPAQVQLVESGGGVVQPGRSLRLSCAASGFTFSSYAMSWVRQAPGKGLEWVSAISGSGDSTYYADSVKGRFTISRDNSKNTLYLQMNSLRAEDTAVYYCAKDLDSSSPDDAFDIWGQGTTVTVSSGGAALAEVAAAVADPIVLTQSPGKLSLSPGERATLSCGASQSVSSNYLAWYQQKPGLAPRLLIYDASIRATGVPDRFSGSGSATDFTLTISRLDPEDFAVYYCHQYSSAPMTFGQGTKLEIKR

SEQ ID NO: 8 - DNA, 783 bp, synthetic construct
  source 1..783: mol_type = other DNA
atggccttaccagtgaccgccttgctcctgccgctggccttgctgctccacgccgccaggccggctcaagtgcagctggtcgaatctggagctgggactaagaagcctggcgagagtctgaagatctcttgtaaggccagcggctacaactttgcttcttactggatcgggtgggttagacagatgcctggaaaaggacttgagtgggtagggatcattgatcctagcgattccgataccaaatacagcccaagctttgaagggcaagtcactattagtgtggacaagtctatcagcactgctcacctgcagtggagtagcctagatgcgagcgacacggccatgtactactgcgcgaggagtctgggcagctactatggagattggtatttcgatctctgggggaggggcacactggtgaccgtgtcatccggaggaggcggcgggtccaactttatgctgacccagccacattcaatgtctgaaagccctggcaaaaccgtaacaatctcgtgcacacggtcaagcgggagcatcgccaacaactatgttcagtggtatcagcagagacccgggagttcccctaccactgtcatctacgatgacaatcagagaccatccggggtgcccgaccgcttcagcggctcaatagatagctccagcaactccgcctctttaacaatctcgggcctgaaaaccgaggacgaagctgattattactgccagtcctatgactccagcaacgtgatttttggtggagggactaaactgaccgtcctcgga

SEQ ID NO: 9 - protein, 261 aa, synthetic construct
MALPVTALLLPLALLLHAARPAQVQLVESGAGTKKPGESLKISCKASGYNFASYWIGWVRQMPGKGLEWVGIIDPSDSDTKYSPSFEGQVTISVDKSISTAHLQWSSLDASDTAMYYCARSLGSYYGDWYFDLWGRGTLVTVSSGGGGGSNFMLTQPHSMSESPGKTVTISCTRSSGSIANNYVQWYQQRPGSSPTTVIYDDNQRPSGVPDRFSGSIDSSSNSASLTISGLKTEDEADYYCQSYDSSNVIFGGGTKLTVLG

SEQ ID NO: 10 - DNA, 858 bp, synthetic construct
  source 1..858: mol_type = other DNA
atggccttaccagtgaccgccttgctcctgccgctggccttgctgctccacgccgccaggccggctcaagttcagctggtgcagagcggagcggaggtgaagaagccagggtcttccgttaaggtctcttgtaaggcctcaggcggaactttctcatcttacgctatttcctgggtaaggcaagcaccaggacagggattagaatggatgggggggattatccccattttcggtaccgcgaactacgcccaaaaatttcaaggtcgggtaaccatcaccgccgatgaatcgacatctaccgcctacatggaactgagctctcttcggagcgaggacaccgcagtctattactgcgcgagagacgggtatagtggtagctactactattattacggcatggacgtttggggccaggggaccttagtgacggtgagcagcggatccccatacattcaaaatccagaacctgcggtataccaattgaaagacccaagaagccaggattccacgctgtgtctgtttacagacttcgactcacagatcaatgttcccaagacaatggaaagcgggacgttcatcaccgataaatgcgttctggacatgaaggctatggattctaaaagtaatggtgctatagcctggagtaaccagacctcatttacttgccaagacattttcaaggagaccaatgctacatatccatcctccgacgtcccttgtgacgccactctgaccgagaaatcttttgagaccgatatgaacctgaactttcagaacctactggttatcgttctgagaatactgttgctcaaagtcgccggattcaaccttctcatgactctgagactgtggagtagc

SEQ ID NO: 11 - protein, 286 aa, synthetic construct
MALPVTALLLPLALLLHAARPAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGYSGSYYYYYGMDVWGQGTLVTVSSGSPYIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 12 - DNA, 921 bp, synthetic construct
  source 1..921: mol_type = other DNA
atggagacagacacactcctgctatgggtgctgctgctctgggttccaggttccacaggtcagtctgctctgacccaacctgccagcgtctccggcagtccaggacaaagtatcaccatcagctgcactgggacttccagcgatgtggggggctacaattacgtaagttggtaccagcaacaccctgggaaggcccccaagctgatgatttatgatgtctcatacagaccgagcggagtgagccaccgttttagtggctccaagtccggaaacaccgcttcactaacaatcagtggcttacaagcagaggacgaagccgattattactgctcatcttacaccagttccagtacgcttgtgtttggtactggaacaaagttgactgtgctgggcacgcgtgaggacttgaggaatgtgacgccaccgaaggtctctttgtttgagccgtctaaggcagaaattgctaataaacaaaaggccaccctagtgtgtctcgcgaggggattctttcctgatcacgttgagttgtcctggtgggtgaatggtaaggaggttcatagcggtgtctgcaccgacccccaggcatacaaggagtcgaactactcctactgcctgtcatctagactccgagtcagtgccacgttctggcataacccccgtaaccattttaggtgccaggtgcaattccacgggcttagtgaagaggacaagtggccagaggggtccccaaagccggtgacccaaaatatctctgcggaagcttgggggcgcgccgattgtgggattaccagcgcgagctatcagcagggagtcctgagtgcaacgatactctacgagatattactgggaaaggctacattatatgcagtgttggtgagcactctggtcgtcatggcaatggttaagagaaagaatagctaa

SEQ ID NO: 13 - protein, 306 aa, synthetic construct
METDTLLLWVLLLWVPGSTGQSALTQPASVSGSPGQSITISCTGTSSDVGGYNYVSWYQQHPGKAPKLMIYDVSYRPSGVSHRFSGSKSGNTASLTISGLQAEDEADYYCSSYTSSSTLVFGTGTKLTVLGTREDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 14 - DNA, 1209 bp, synthetic construct
  source 1..1209: mol_type = other DNA
atggccttaccagtgaccgccttgctcctgccgctggccttgctgctccacgccgccaggccggctcaagttcagctggtgcagagcggagcggaggtgaagaagccagggtcttccgttaaggtctcttgtaaggcctcaggcggaactttctcatcttacgctatttcctgggtaaggcaagcaccaggacagggattagaatggatgggggggattatccccattttcggtaccgcgaactacgcccaaaaatttcaaggtcgggtaaccatcaccgccgatgaatcgacatctaccgcctacatggaactgagctctcttcggagcgaggacaccgcagtctattactgcgcgagagacgggtatagtggtagctactactattattacggcatggacgtttggggccaggggaccttagtgacggtgagcagcggaggaggcggcgggtcccagtctgctctgacccaacctgccagcgtctccggcagtccaggacaaagtatcaccatcagctgcactgggacttccagcgatgtggggggctacaattacgtaagttggtaccagcaacaccctgggaaggcccccaagctgatgatttatgatgtctcatacagaccgagcggagtgagccaccgttttagtggctccaagtccggaaacaccgcttcactaacaatcagtggcttacaagcagaggacgaagccgattattactgctcatcttacaccagttccagtacgcttgtgtttggtactggaacaaagttgactgtgctgggcggatccccatacattcaaaatccagaacctgcggtataccaattgaaagacccaagaagccaggattccacgctgtgtctgtttacagacttcgactcacagatcaatgttcccaagacaatggaaagcgggacgttcatcaccgataaatgcgttctggacatgaaggctatggattctaaaagtaatggtgctatagcctggagtaaccagacctcatttacttgccaagacattttcaaggagaccaatgctacatatccatcctccgacgtcccttgtgacgccactctgaccgagaaatcttttgagaccgatatgaacctgaactttcagaacctactggttatcgttctgagaatactgttgctcaaagtcgccggattcaaccttctcatgactctgagactgtggagtagc

SEQ ID NO: 15 - protein, 403 aa, synthetic construct
MALPVTALLLPLALLLHAARPAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGYSGSYYYYYGMDVWGQGTLVTVSSGGGGGSQSALTQPASVSGSPGQSITISCTGTSSDVGGYNYVSWYQQHPGKAPKLMIYDVSYRPSGVSHRFSGSKSGNTASLTISGLQAEDEADYYCSSYTSSSTLVFGTGTKLTVLGGSPYIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 16 - DNA, 1323 bp, synthetic construct
  source 1..1323: mol_type = other DNA
atggccttaccagtgaccgccttgctcctgccgctggccttgctgctccacgccgccaggccggctcaagttcagctggtgcagagcggagcggaggtgaagaagccagggtcttccgttaaggtctcttgtaaggcctcaggcggaactttctcatcttacgctatttcctgggtaaggcaagcaccaggacagggattagaatggatgggggggattatccccattttcggtaccgcgaactacgcccaaaaatttcaaggtcgggtaaccatcaccgccgatgaatcgacatctaccgcctacatggaactgagctctcttcggagcgaggacaccgcagtctattactgcgcgagagacgggtatagtggtagctactactattattacggcatggacgtttggggccaggggaccttagtgacggtgagcagcggaggaggcggcgggtcccagtctgctctgacccaacctgccagcgtctccggcagtccaggacaaagtatcaccatcagctgcactgggacttccagcgatgtggggggctacaattacgtaagttggtaccagcaacaccctgggaaggcccccaagctgatgatttatgatgtctcatacagaccgagcggagtgagccaccgttttagtggctccaagtccggaaacaccgcttcactaacaatcagtggcttacaagcagaggacgaagccgattattactgctcatcttacaccagttccagtacgcttgtgtttggtactggaacaaagttgactgtgctgggcggatccacgcgtgaggacttgaggaatgtgacgccaccgaaggtctctttgtttgagccgtctaaggcagaaattgctaataaacaaaaggccaccctagtgtgtctcgcgaggggattctttcctgatcacgttgagttgtcctggtgggtgaatggtaaggaggttcatagcggtgtctgcaccgacccccaggcatacaaggagtcgaactactcctactgcctgtcatctagactccgagtcagtgccacgttctggcataacccccgtaaccattttaggtgccaggtgcaattccacgggcttagtgaagaggacaagtggccagaggggtccccaaagccggtgacccaaaatatctctgcggaagcttgggggcgcgccgattgtgggattaccagcgcgagctatcagcagggagtcctgagtgcaacgatactctacgagatattactgggaaaggctacattatatgcagtgttggtgagcactctggtcgtcatggcaatggttaagagaaagaatagctaa

SEQ ID NO: 17 - protein, 440 aa, synthetic construct
MALPVTALLLPLALLLHAARPAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGYSGSYYYYYGMDVWGQGTLVTVSSGGGGGSQSALTQPASVSGSPGQSITISCTGTSSDVGGYNYVSWYQQHPGKAPKLMIYDVSYRPSGVSHRFSGSKSGNTASLTISGLQAEDEADYYCSSYTSSSTLVFGTGTKLTVLGGSTREDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 18 - DNA, 1527 bp, synthetic construct
  source 1..1527: mol_type = other DNA
atggattggatctggcgcatcctgtttctcgtgggagccgccacaggcgcccattctgctcaagttcagctggtgcagagcggagcggaggtgaagaagccagggtcttccgttaaggtctcttgtaaggcctcaggcggaactttctcatcttacgctatttcctgggtaaggcaagcaccaggacagggattagaatggatgggggggattatccccattttcggtaccgcgaactacgcccaaaaatttcaaggtcgggtaaccatcaccgccgatgaatcgacatctaccgcctacatggaactgagctctcttcggagcgaggacaccgcagtctattactgcgcgagagacgggtatagtggtagctactactattattacggcatggacgtttggggccaggggaccttagtgacggtgagcagcggaggaggcggcgggtcccagtctgctctgacccaacctgccagcgtctccggcagtccaggacaaagtatcaccatcagctgcactgggacttccagcgatgtggggggctacaattacgtaagttggtaccagcaacaccctgggaaggcccccaagctgatgatttatgatgtctcatacagaccgagcggagtgagccaccgttttagtggctccaagtccggaaacaccgcttcactaacaatcagtggcttacaagcagaggacgaagccgattattactgctcatcttacaccagttccagtacgcttgtgtttggtactggaacaaagttgactgtgctgggctccggaggtggtggatccgatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcaggtggtggtggttctggcggcggcggctccggtggtggtggttctgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaatcctaa

SEQ ID NO: 19 - protein, 508 aa, synthetic construct
MDWIWRILFLVGAATGAHSAQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGYSGSYYYYYGMDVWGQGTLVTVSSGGGGGSQSALTQPASVSGSPGQSITISCTGTSSDVGGYNYVSWYQQHPGKAPKLMIYDVSYRPSGVSHRFSGSKSGNTASLTISGLQAEDEADYYCSSYTSSSTLVFGTGTKLTVLGSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS

SEQ ID NO: 20 - DNA, 648 bp, synthetic construct
  source 1..648: mol_type = other DNA
atggagacagacacactcctgctatgggtgctgctgctctgggttccaggttccacaggtggcggcggtgggtcagaacaaaaattgatttcagaggaagatctcggagggggagggtcaacgcgtgaggacttgaggaatgtgacgccaccgaaggtctctttgtttgagccgtctaaggcagaaattgctaataaacaaaaggccaccctagtgtgtctcgcgaggggattctttcctgatcacgttgagttgtcctggtgggtgaatggtaaggaggttcatagcggtgtctgcaccgacccccaggcatacaaggagtcgaactactcctactgcctgtcatctagactccgagtcagtgccacgttctggcataacccccgtaaccattttaggtgccaggtgcaattccacgggcttagtgaagaggacaagtggccagaggggtccccaaagccggtgacccaaaatatctctgcggaagcttgggggcgcgccgattgtgggattaccagcgcgagctatcagcagggagtcctgagtgcaacgatactctacgagatattactgggaaaggctacattatatgcagtgttggtgagcactctggtcgtcatggcaatggttaagagaaagaatagctaa

SEQ ID NO: 21 - protein, 215 aa, synthetic construct
METDTLLLWVLLLWVPGSTGGGGGSEQKLISEEDLGGGGSTREDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 22 - protein, 125 aa, synthetic construct
AQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDGYSGSYYYYYGMDVWGQGTLVTVSS

SEQ ID NO: 23 - protein, 123 aa, synthetic construct
AQVQLVESGGGVVQPGRSLRLSCAASGFTFSSYAMSWVRQAPGKGLEWVSAISGSGDSTYYADSVKGRFTISRDNSKNTLYLQMNSLRAEDTAVYYCAKDLDSSSPDDAFDIWGQGTTVTVSS

SEQ ID NO: 24 - protein, 123 aa, synthetic construct
AQVQLVESGAGTKKPGESLKISCKASGYNFASYWIGWVRQMPGKGLEWVGIIDPSDSDTKYSPSFEGQVTISVDKSISTAHLQWSSLDASDTAMYYCARSLGSYYGDWYFDLWGRGTLVTVSS

SEQ ID NO: 25 - protein, 110 aa, synthetic construct
QSALTQPASVSGSPGQSITISCTGTSSDVGGYNYVSWYQQHPGKAPKLMIYDVSYRPSGVSHRFSGSKSGNTASLTISGLQAEDEADYYCSSYTSSSTLVFGTGTKLTVL

SEQ ID NO: 26 - protein, 121 aa, synthetic construct
AALAEVAAAVADPIVLTQSPGKLSLSPGERATLSCGASQSVSSNYLAWYQQKPGLAPRLLIYDASIRATGVPDRFSGSGSATDFTLTISRLDPEDFAVYYCHQYSSAPMTFGQGTKLEIKR

SEQ ID NO: 27 - protein, 111 aa, synthetic construct
NFMLTQPHSMSESPGKTVTISCTRSSGSIANNYVQWYQQRPGSSPTTVIYDDNQRPSGVPDRFSGSIDSSSNSASLTISGLKTEDEADYYCQSYDSSNVIFGGGTKLTVLG

SEQ ID NO: 28 - protein, 8 aa, synthetic construct
GGTFSSYA

SEQ ID NO: 29 - protein, 8 aa, synthetic construct
IIPIFGTA

SEQ ID NO: 30 - protein, 17 aa, synthetic construct
ARDGYSGSYYYYYGMDV

SEQ ID NO: 31 - protein, 9 aa, synthetic construct
SSDVGGYNY

SEQ ID NO: 32 - skipped

SEQ ID NO: 33 - protein, 10 aa, synthetic construct
SSYTSSSTLV

SEQ ID NO: 34 - protein, 8 aa, synthetic construct
GFTFSSYA

SEQ ID NO: 35 - protein, 8 aa, synthetic construct
ISGSGDST

SEQ ID NO: 36 - protein, 15 aa, synthetic construct
AKDLDSSSPDDAFDI

SEQ ID NO: 37 - protein, 7 aa, synthetic construct
QSVSSNY

SEQ ID NO: 38 - skipped

SEQ ID NO: 39 - protein, 9 aa, synthetic construct
HQYSSAPMT

SEQ ID NO: 40 - protein, 8 aa, synthetic construct
GYNFASYW

SEQ ID NO: 41 - protein, 8 aa, synthetic construct
IDPSDSDT

SEQ ID NO: 42 - protein, 15 aa, synthetic construct
ARSLGSYYGDWYFDL

SEQ ID NO: 43 - protein, 8 aa, synthetic construct
SGSIANNY

SEQ ID NO: 44 - skipped

SEQ ID NO: 45 - protein, 9 aa, synthetic construct
QSYDSSNVI

SEQ ID NO: 46 - protein, 40 aa, synthetic construct
  VARIANT 1..40: note = GGGS repeats may be deleted
GGGSGGGSGGGSGGGSGGGSGGGSGGGSGGGSGGGSGGGS

SEQ ID NO: 47 - protein, 50 aa, synthetic construct
  VARIANT 1..50: note = GGGGS repeats may be deleted
GGGGSGGGGSGGGGSGGGGSGGGGSGGGGSGGGGSGGGGSGGGGSGGGGS

SEQ ID NO: 48 - protein, 8 aa, synthetic construct
GGGSGGGS

SEQ ID NO: 49 - protein, 14 aa, synthetic construct
GGGSGGGGSGGGGS

(19) United States
(12) Patent Application Publication
(10) Pub. No.: US 2025/0250359 A1
(43) Pub. Date: Aug. 7, 2025

(54) TERMINAL DEOXYNUCLEOTIDYL TRANSFERASE ANTIBODIES AND USES THEREOF

(71) Applicants: Baylor College of Medicine, Houston, TX (US); The Scripps Research Institute, La Jolla, CA (US)

(72) Inventors: Maksim Mamonkin, Houston, TX (US); Norihiro Watanabe, Houston, TX (US); Alexey Stepanov, La Jolla, CA (US); Ding Zhang, La Jolla, CA (US)

(73) Assignees: Baylor College of Medicine, Houston, TX (US); The Scripps Research Institute, La Jolla, CA (US)

(21) Appl. No.: 18/859,245

(22) PCT Filed: Apr. 28, 2023
(86) PCT No.: PCT/US2023/066341

§ 371 Date: Oct. 23, 2024

Related U.S. Application Data
(60) Provisional application No. 63/336,912, filed on Apr. 29, 2022.

(51) Int. Cl. C07K 16/40; C07K 16/28; C12N 5/071; C12N 15/11

(52) U.S. Cl. CPC C07K 16/40; C07K 16/283; C12N 5/0602; C12N 15/11; C07K 2317/24; C07K 2317/31; C07K 2317/35; C07K 2317/565; C07K 2317/62; C07K 2317/92; C12N 2510/00

(57) ABSTRACT

Aspects of the disclosure relate to novel antibody and antigen binding fragments. Further aspects relate to polypeptides comprising the antigen binding fragment(s) of the disclosure, and compositions comprising the polypeptides, antibodies, and/or antigen binding fragments of the disclosure. Also described are nucleic acids encoding an antibody or antigen binding fragment of the disclosure. In particular embodiments, the disclosure provides immunological compositions that target TdT peptides, including those associated with HLA-A2. Specific embodiments allow for detection, diagnosis, treatment, and prevention of cancer in an individual in need thereof.

This application claims priority to U.S. Provisional Patent Application Ser. No. 63/336,912, filed Apr. 29, 2022, which is incorporated by reference herein in its entirety.

### BACKGROUND

### I. Technical Field

Aspects of the disclosure relate at least to the fields of immunology, cell biology, molecular biology, and medicine.

### II. Background

Terminal deoxynucleotidyl transferase (TdT) is a DNA polymerase expressed in T- and B-cell progenitors where it catalyzes transfer of nucleotides during DNA rearrangement (Kunkel et al., 1986; Desiderio et al., 1984). TdT is expressed in up to 95% of T- and B-lineage ALL and is widely used as a diagnostic marker for these malignancies (Drexler et al., 1986). In addition, TdT expression is frequently detected in minimally differentiated AML (Patel et al., 2012; Drexler et al, 1993). Off-target activity of TdT in immature progenitors has been linked with leukemogenesis (Borrow et al., 2019a; Borrow et al., 2019b). Absence of TdT expression in primitive hematopoietic progenitors or mature peripheral lymphocytes makes it an ideal therapeutic target for leukemia.

### SUMMARY

Aspects of the disclosure relate to novel antibody and antigen binding fragments, as well as methods of using these antibodies and functional fragments. Further aspects relate to polypeptides of any kind comprising the antigen binding fragment(s) of the disclosure, and compositions comprising the polypeptides, antibodies, and/or antigen binding fragments of the disclosure. Also described are nucleic acids encoding an antibody or antigen binding fragment of the disclosure. The disclosure also relates to nucleic acids encoding an antibody heavy chain, wherein the nucleic acid has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS:22-24. Also described are nucleic acids encoding an antibody light chain of the disclosure, wherein the nucleic acid has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to one of SEQ ID NOS: 25-27. Further aspects relate to vectors or expression vectors comprising nucleic acids of the disclosure and host cells comprising polypeptides, nucleic acids, vectors, antibodies, or antigen binding fragments of the disclosure. The nucleic acids of the disclosure may be DNA or RNA.

Also described is a method of a making a cell comprising transferring one or more nucleic acid(s) of the disclosure into a cell. In some embodiments, the method further comprises culturing the cell under conditions that allow for expression of a polypeptide from the nucleic acid. In some embodiments, the method further comprising isolating the expressed polypeptide. The cell may be further defined as a human cell, immune effector cell, B cell, T cell, Chinese hamster ovary, NS0 murine myeloma cell, PER.C6 cell, or any cell described herein.

Further aspects of the disclosure relate to a method for treating or preventing a cancer in a subject, the method comprising administering to the subject an antibody or composition comprising same, antigen binding fragment or composition comprising same, polypeptide, nucleic acid, or host cell of the disclosure. Yet further aspects relate to a method for evaluating a sample from a subject, the method comprising contacting a biological sample from the subject, or extract thereof, with at least one antibody, antigen binding fragment, or polypeptide of the disclosure. Also disclosed is a method for diagnosing a cancer in a subject, the method comprising contacting a biological sample from the subject, or extract thereof, with at least one antibody, antigen binding fragment, or polypeptide of any one of the disclosure. In some aspects, the compositions of the disclosure are formulated as a vaccine for the treatment or prevention of cancer. In some embodiments, the antibodies, antigen binding fragments, or compositions of the disclosure are used in a vaccine for preventing cancer in a subject that does not have cancer. In some embodiments, the antibodies, antigen binding fragments, or compositions of the disclosure are used to treat a subject having cancer.

Also described is a method of a making a cell comprising transferring one or more nucleic acid(s) of the disclosure into a cell. In some aspects, the method further comprises culturing the cell under conditions that allow for expression of a polypeptide from the nucleic acid. In some aspects, the method further comprising isolating the expressed polypeptide. Aspects describe a method for producing a polypeptide comprising transferring one or more nucleic acid(s) or vector(s) of the disclosure into a cell and isolating polypeptides expressed from the nucleic acid. The cell may be further defined as a human cell, immune effector cell, B cell, T cell, Chinese hamster ovary, NS0 murine myeloma cell, PER.C6 cell, or any cell described herein.

Further aspects of the disclosure relate to a method for treating or preventing cancer in a subject, the method comprising administering to the subject an antibody or composition comprising same, antigen binding fragment or composition comprising same, polypeptide, nucleic acid, or host cell of the disclosure. Yet further aspects relate to a method for evaluating a sample from a subject, the method comprising contacting a biological sample from the subject, or extract thereof, with at least one antibody or composition comprising same, antigen binding fragment or composition comprising same, or polypeptide of the disclosure. Also disclosed is a method for diagnosing cancer in a subject, the method comprising contacting a biological sample from the subject, or extract thereof, with at least one antibody or composition comprising same, antigen binding fragment or composition comprising same, or polypeptide of any one of the disclosure. In some aspects, the compositions of the disclosure are formulated as a vaccine for the treatment or prevention of cancer. In some aspects, the antibodies or composition comprising same, antigen binding fragments or composition comprising same, or compositions of the disclosure are used in a vaccine for preventing cancer in a subject that does not have cancer. In some aspects, the antibodies or composition comprising same, antigen binding fragments or composition comprising same, or compositions of the disclosure are used to treat a subject having cancer.

Certain aspects of the disclosure relate to an antibody or antigen binding fragment comprising a heavy chain variable region and a light chain variable region, wherein the heavy chain variable region comprises a particular sequence and wherein the light chain variable region comprises a particular sequence. Further aspects relate to an antibody or antigen binding fragment comprising a heavy chain variable region and a light chain variable region, wherein the heavy chain variable region comprises at least 80% sequence identity or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with SEQ ID NO:22-24. Certain aspects relate to an antibody or antigen binding fragment comprising a heavy chain variable region and a light chain variable region, wherein the light chain variable region comprises at least 80% sequence identity or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with SEQ ID NO:25-27.

Aspects of the disclosure relate to an antibody or antigen binding fragment comprising a heavy chain variable region and a light chain variable region, wherein the heavy chain variable region comprises a HCDR1, HCDR2, and HCDR3 from a heavy chain variable region of an antibody clone from B1, G7, or G9 and wherein the light chain variable region comprises a LCDR1, LCDR2, and LCDR3 from the light chain variable region of the same respective clone of B1, G7, or G9. Further aspects relate to an antibody or antigen binding fragment comprising a heavy chain variable region and a light chain variable region, wherein the heavy chain variable region comprises a HCDR1, HCDR2, and HCDR3 having or having at least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a HCDR1, HCDR2, and HCDR3 from a heavy chain variable region of an antibody clone of B1, G7, or G9, and wherein the light chain variable region comprises a LCDR1, LCDR2, and LCDR3 having or having at least least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a LCDR1, LCDR2, and LCDR3 from the light chain variable region of the same respective clone of B1, G7, or G9.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:40-42, respectively and the LCDR1, LCDR2, and LCDR3 comprises an amino acid sequence of SEQ ID NOS:43, 45 or “DDN”, respectively.

The HCDR1, HCDR2, HCDR3, LCDR1, LCDR2, and/or LCDR3 may be determined from the variable region sequences by methods known in the art. In some aspects, the CDR is HCDR1, HCDR2, HCDR3, LCDR1, LCDR2, and/or LCDR3 determined by the Chothia method. In some aspects, the CDR is HCDR1, HCDR2, HCDR3, LCDR1, LCDR2, and/or LCDR3 determined by the Kabat method. In some aspects, the CDR is HCDR1, HCDR2, HCDR3, LCDR1, LCDR2, and/or LCDR3 determined by the IMGT method.

The polypeptides of the disclosure may comprise at least two antigen binding fragments, wherein each antigen binding fragment is independently selected from an antigen binding fragment of the disclosure. In some aspects, the polypeptide is multivalent. In some aspects, the polypeptide is multispecific. In some aspects, the polypeptide is bispecific. In some aspects, the polypeptide comprises, comprises at least, or comprises at most 1, 2, 3, 4, 5, 6, 7, 8, 9, or 10 antigen binding regions. Each antigen binding region may be independently selected from an antigen binding region of the disclosure. In some aspects, the polypeptide may have repeated units of the same antigen binding region, such as at least, at most, or exactly 1, 2, 3, 4, 5, 6, 7, 8, 9, or 10 repeated units.

In some aspects, the heavy chain variable region comprises an amino acid sequence with at least 80% sequence identity to a heavy chain variable region of an antibody clone B1,G7, or G9 and/or the light chain variable region comprises an amino acid sequence with at least 80% sequence identity to the light chain variable region of an antibody clone B1, G7, or G9. In some aspects, the heavy chain variable region comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain variable region of an antibody clone B1 and/or the light chain variable region comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the light chain variable region of the same antibody clone B1. In some aspects, the heavy chain variable region comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain variable region of an antibody clone G7 and/or the light chain variable region comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the light chain variable region of the same antibody clone G7. In some aspects, the heavy chain variable region comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain variable region of an antibody clone G9 and/or the light chain variable region comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to the light chain variable region of the same antibody clone G9.

In some aspects, the heavy chain variable region comprises a heavy chain framework region that has or has at least 80% sequence identity to a heavy chain framework region of an antibody clone B1 and the light chain variable region comprises a light chain framework region that has or has at least 80% sequence identity to a light chain framework region of the same antibody clone B1. In some aspects, the heavy chain variable region comprises a heavy chain framework region having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain framework region of an antibody clone B1 and the light chain variable region comprises a light chain framework region having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a light chain framework region of the same antibody clone B1.

In some aspects, the heavy chain variable region comprises a heavy chain framework region that has or has at least 80% sequence identity to a heavy chain framework region of an antibody clone G7 and the light chain variable region comprises a light chain framework region that has or has at least 80% sequence identity to a light chain framework region of the same antibody clone G7. In some aspects, the heavy chain variable region comprises a heavy chain framework region having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain framework region of an antibody clone G7 and the light chain variable region comprises a light chain framework region having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a light chain framework region of the same antibody clone G7.

In some aspects, the heavy chain variable region comprises a heavy chain framework region that has or has at least 80% sequence identity to a heavy chain framework region of an antibody clone G9 and the light chain variable region comprises a light chain framework region that has or has at least 80% sequence identity to a light chain framework region of the same antibody clone G9. In some aspects, the heavy chain variable region comprises a heavy chain framework region having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a heavy chain framework region of an antibody clone G9 and the light chain variable region comprises a light chain framework region having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity to a light chain framework region of the same antibody clone G9.

The antibody or antigen binding fragment of the disclosure may be human, chimeric, or humanized. In some aspects, the antibody, or antigen binding fragment binds a TdT peptide with a kD of about 10⁻³ M to about 10⁻⁹ M. In some aspects, the antibody, or antigen binding fragment binds a a TdT peptide with a kD of about, a kD of at least, or a kD of at most 10⁻³, 10⁻⁴, 10⁻⁵, 10⁻⁶, 10⁻⁷, 10⁻⁸, 10⁻⁹, 10⁻¹⁰, 10⁻¹¹, 10⁻¹², 10⁻¹³, 10⁻¹⁴, 10⁻¹⁵, 10⁻¹⁶, 10⁻¹⁷, or 10⁻¹⁸ (or any derivable range therein) M. In some aspects, the antibody or antigen binding fragment specifically binds to a TdT peptide. The antibody may be further defined as a neutralizing antibody. In some aspects, the antibody or antigen binding fragment is further defined as a human antibody or antigen binding fragment, humanized antibody or antigen binding fragment, recombinant antibody or antigen binding fragment, chimeric antibody or antigen binding fragment, an antibody or antigen binding fragment derivative, a veneered antibody or antigen binding fragment, a diabody, a monoclonal antibody or antigen binding fragment, a single domain antibody, or a single chain antibody. In some aspects, the antigen binding fragment is further defined as a single chain variable fragment (scFv), F(ab′)₂, Fab′, Fab, Fv, or rIgG. In some aspects, the antibody, antigen binding fragment, or polypeptide is operatively linked to a detectable label. Detectable labels are encompassed herein.

Aspects of the disclosure also relate to multi-specific and/or multivalent antibodies and polypeptides. Accordingly, aspects relate to bivalent or bispecific antibodies that comprise two antigen binding fragments, wherein the antigen binding fragment is two of the same antigen binding fragments or two different antigen binding fragments described herein. The disclosure also provides for multi-specific polypeptides. Aspects relate to polypeptides comprising or comprising at least 2, 3, 4, 5, or 6 antigen binding fragments.

The antigen binding fragment may be at least 2, 3, 4, 5, or 6 scFv, F(ab′)₂, Fab′, Fab, Fv, or rIgG, or combinations thereof. The polypeptide and/or antigen binding fragments of the disclosure may comprise a linker between a heavy chain and light chain variable region or between antigen binding fragments. The linker may be a flexible linker. Exemplary flexible linkers include glycine polymers (G)n or glycine-serine polymers. Glycine-alanine polymers, alanine-serine polymers, and other flexible linkers known in the art and may be used as a linker in the polypeptides of the disclosure. Specific examples include (GGGS)n (SEQ ID NO: 46) or (GGGGS)n (SEQ ID NO: 47), where n=1-10, and further specific examples include GGGSGGGS (SEQ ID NO: 48) or GGGSGGGGSGGGGS (SEQ ID NO: 49).

Compositions of the disclosure, such as pharmaceutical compositions may comprise a pharmaceutical excipient, carrier, or molecule described herein. In some aspects, the composition further comprises an adjuvant or an immunostimulator. Such adjuvants or immunostimulators may include, but are not limited to stimulators of pattern recognition receptors, such as Toll-like receptors, RIG-1 and NOD-like receptors (NLR), mineral salts, such as alum, alum combined with monphosphoryl lipid (MPL) A of Enterobacteria, such as Escherihia coli, Salmonella minnesota, Salmonella typhimurium, or Shigella flexneri or specifically with MPL (AS04), MPL A of above-mentioned bacteria separately, saponins, such as QS-21, Quil-A, ISCOMs, ISCOMATRIX, emulsions such as MF59, Montanide, ISA 51 and ISA 720, AS02 (QS21+squalene+MPL.), liposomes and liposomal formulations such as AS01, synthesized or specifically prepared microparticles and microcarriers such as bacteria-derived outer membrane vesicles (OMV) of N. gonorrheae, Chlamydia trachomatis and others, or chitosan particles, depot-forming agents, such as Pluronic block co-polymers, specifically modified or prepared peptides, such as muramyl dipeptide, aminoalkyl glucosaminide 4-phosphates, such as RC529, or proteins, such as bacterial toxoids or toxin fragments. Compositions may comprise more than one antibody and/or antigen binding fragment of the disclosure. Accordingly, compositions of the disclosure may comprise, may comprise at least, or may comprise at most 1, 2, 3, 4, 5, 6, 7, 8, 9, or 10 antibodies and/or antigen binding fragments of the disclosure. The compositions of the disclosure may be formulated for a route of administration described herein. In some aspects, the composition, antibody, antigen binding fragment, or polypeptide is formulated for parenteral, intravenous, subcutaneous, intramuscular, or intranasal administration. In a particular aspect, the compositions is formulated for intranasal administration.

In some aspects, the host cell is a human cell, immune effector cell, immune cell, B cell, T cell, Chinese hamster ovary, NS0 murine myeloma cell, or PER.C6 cell. In some aspects, the host cell is a cell type or cell population described herein.

In aspects of the disclosure, the subject or patient may be a human subject or a human patient. In some aspects, the subject or patient is a non-human animal. In some aspects, the non-human animal is a bat, monkey, camel, rat, mouse, rabbit, goat, chicken, bird, cat, horse, or dog. The subject may further be defined as an at-risk subject, compared to the general population. At-risk subjects include those individuals that may be smokers, have a personal or family history of cancer, have exposure to carcinogens, are greater than 50, 55, 60, 65, or 70 years in age, and so forth. In some aspects, the subject has one or more symptoms of cancer. Examples of cancer symptoms include fatigue or extreme tiredness that does not improve with rest; weight loss or gain of 10 pounds or more for no known reason; eating problems such as not feeling hungry, trouble swallowing, belly pain, or nausea and vomiting; swelling or lumps anywhere in the body; thickening or lump in the breast or other part of the body; pain, especially new or with no known reason, that does subside or that worsens; skin changes such as a lump that bleeds or turns scaly, a new mole or a change in a mole, a sore that does not heal, or a yellowish color to the skin or eyes (jaundice); cough or hoarseness that does not go away; unusual bleeding or bruising for no known reason; change in bowel habits, such as constipation or diarrhea, that does not subside or a change in how the stools look; bladder changes such as pain when passing urine, blood in the urine or needing to pass urine more or less often; fever or nights sweats; headaches; and/or vision or hearing problems. In some aspects, the subject does not have any symptom of cancer. In some aspects, the subject has been diagnosed with cancer. In some aspects, the subject has not been diagnosed with cancer. In some aspects, the subject has been previously treated for cancer. In some aspects, the subject has not been previously vaccinated for cancer. In some aspects, the previous treatment comprises surgery, radiation, chemotherapy, drug therapy, hormone therapy, or a combination thereof.

In some aspects of the disclosure, the method further comprises incubating the antibody, antigen binding fragment, or polypeptide under conditions that allow for the binding of the antibody, antigen binding fragment, or polypeptide to antigens in a biological sample or extract thereof. In some aspects, the method further comprises detecting the binding of an antigen to the antibody, antigen binding fragment, or polypeptide. In some aspects, the method further comprises contacting a biological sample with at least one capture antibody, antigen, or polypeptide. The at least one capture antibody, antigen binding fragment, or polypeptide may be an antibody, polypeptide, or antigen binding fragment of the disclosure. In some aspects, the capture antibody is linked or operatively linked to a solid support. The term “operatively linked” refers to a situation where two components are combined or capable of combining to form a complex. For example, the components may be covalently attached and/or on the same polypeptide, such as in a fusion protein or the components may have a certain degree of binding affinity for each other, such as a binding affinity that occurs through van der Waals forces. In some aspects, the biological sample comprises a blood sample, urine sample, fecal sample, nasopharyngeal sample, cerebrospinal fluid sample, cheek scraping sample, nipple aspirate sample, biopsy sample, or a combination thereof. In aspects of the disclosure, the at least one antibody, antigen binding fragment, or polypeptide may be operatively linked to a detectable label. In some aspects, the method further comprises incubating the antibody, antigen binding fragment, or polypeptide under conditions that allow for the binding of the antibody, antigen binding fragment, or polypeptide to antigens in the biological sample or extract thereof. In some aspects, the method further comprises detecting the binding of an antigen to the antibody, antigen binding fragment, or polypeptide. In some aspects, the method further comprises contacting a biological sample with at least one capture antibody, antigen, or polypeptide.

Aspects of the disclosure relate to an antibody, antigen binding fragment, or polypeptide comprising a heavy chain variable region and a light chain variable region of SEQ ID NOS:22 and 25, respectively. Aspects of the disclosure relate to an antibody, antigen binding fragment, or polypeptide comprising a heavy chain variable region and a light chain variable region of SEQ ID NOS:23 and 26, respectively. Aspects of the disclosure relate to an antibody, antigen binding fragment, or polypeptide comprising a heavy chain variable region and a light chain variable region of SEQ ID NOS:24 and 27, respectively.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. TdT expression in normal and malignant lymphocytes.

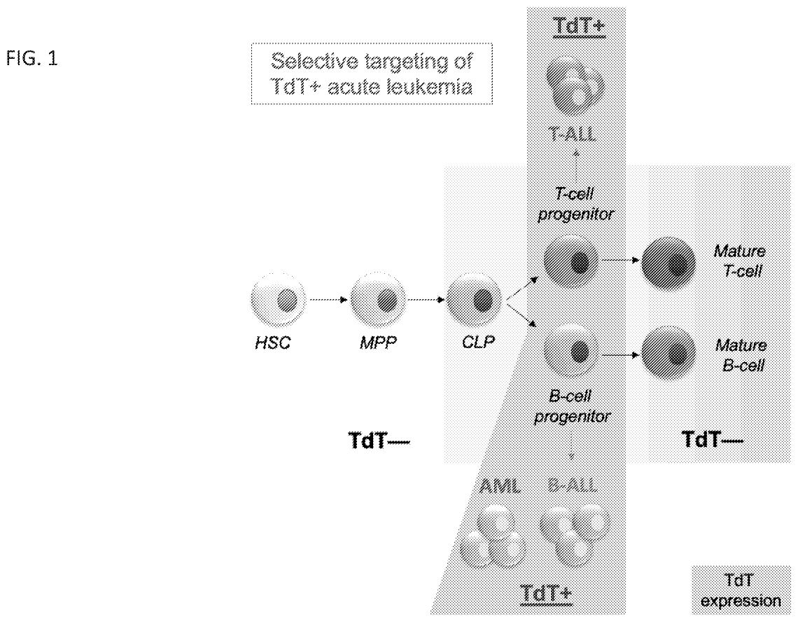

FIG. 2. Targeting TdT with chimeric TCR and BiTE.

FIGS. 3A-3E. Structure, expression, and function of TdT-specific CAR and cTCR. (FIG. 3A) A schematic showing CAR and cTCR structure. (FIG. 3B) Expression of TdT.CAR and cTCR in primary T-cells (n=7 donors). (FIG. 3C) Expansion of modified T-cells post-transduction (n=7). (FIG. 3D) Expression of CD27 and CD45RA on control and TdT-specific CD8+ T cells (n=6). (FIG. 3E) Cytotoxicity of TdT-specific T cells against BV173 cells upon a 3-day coculture at a 1:4 E:T ratio (n=3).

FIGS. 4A-4B. Specificity of TdT.cTCR T-cells. (FIG. 4A) Cytotoxicity of cTCR T cells against BV173 (TdT+/A2+), NALM6 (TdT+/A2+), CCRF-CEM (TdT+/A2-), and THP-1 (TdT-/A2+) cells upon a 3-day coculture. (n=3-4) (FIG. 4B) Viability of normal autologous T and B-cells after a coculture with TdT.cTCR- or CD19.CAR-expressing T-cells measured by Annexin V/7-AAD staining (n=3).

FIGS. 5A-5B. Effector function of TdT.cTCR-transduced CD4+ and CD8+ T-cells. Representative flow plots showing degranulation (FIG. 5A), and IFNγ/TNFα production (FIG. 5B) upon coculture with parental and MHC I-deficient (b2MKO) BV173 leukemia cells.

FIG. 6. Cytotoxicity of T cells redirected against TdT+/A2+ cell lines through CAR, cTCR, or BiTE. (n=3).

FIG. 7 illustrates one embodiment of a chimeric TCR in which V_{H} and V_{L} domains of the antibody are present on a single TCR α chain, in contrast to other receptors in the art (Liu et al., Sci Transl Med. 2021; 13(586):eabb5191). Murine constant chains of TCRα and TCRβ are illustrated.

FIG. 8 represents cTCR and STAR expression on T cells after retroviral transduction. NT; non-transduced.

FIG. 9 demonstrates cytotoxicity of cTCR and STAR-expressing T cells against BV173 cell line (TdT+, HLA-A2+).

FIG. 10 shows cTCR and BiTE expression on T cells after retroviral transduction. NT; non-transduced.

FIG. 11 demonstrates cytotoxicity of cTCR T cells and BiTE-mediated killing against BV173 cell line (TdT+, HLA-A2+).

### DETAILED DESCRIPTION

### I. Examples of Definitions

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in various embodiments.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

The term “peptide,” as used herein, generally refers to amino acids linked by peptide bonds. Peptides can include amino acid chains between about 5 and 50 residues. Peptides can include amino acid chains shorter than 10 residues, including, oligopeptides, dipeptides, tripeptides, and tetrapeptides. Peptides can include chains longer than 50 residues and may be referred to as “polypeptides” or “proteins.” In specific embodiments, the term refers to the TdT peptide of ALYDKTKRI (SEQ ID NO:3) or any peptide that is at least 70, 75, 80, 85, 86, 87, 88, 89, 90 or greater % identity to SEQ ID NO:3. The peptide may be at least 5, 6, 7, 8, or all of the contiguous residues of SEQ ID NO:3. There may be one or two substitutions compared to SEQ ID NO:3. In particular embodiments, any antibody encompassed herein may be able to target these variant peptides.

The term “subject,” as used herein, generally refers to an animal, such as a mammal (e.g., human). For example, the subject can include a vertebrate, a mammal, a rodent (e.g., a mouse), a primate, a simian or a human. Animals may include, but are not limited to, farm animals, sport animals, and pets, including cats, dogs, and horses. A subject can include a healthy or asymptomatic individual, an individual that has or is suspected of having a disease (e.g., cancer) or a pre-disposition to the disease, and/or an individual that is in need of therapy or suspected of needing therapy. A subject can be a patient. “Individual, “subject,” and “patient” are used interchangeably and can refer to a human or non-human.

“Treating” or treatment of a disease or condition refers to executing a protocol, which may include administering one or more drugs to an individual, such as a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutically effective” as used throughout this application refers to anything that promotes or enhances the well-being of the subject with respect to the medical treatment of this condition. This includes, but is not limited to, a reduction in the frequency or severity of one or more signs or symptoms of a disease and/or a delay in its onset or spreading, including cancer.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Brief Summary, Detailed Description, Claims, and description of Brief Description of the Drawings.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

### II. TdT Peptide-Specific Antibodies

Aspects of the disclosure relate to antibodies, antigen binding fragments thereof, or polypeptides capable of specifically binding to a TdT peptide. Certain aspects relate to antibodies, or fragments thereof, that specifically bind to a TdT peptide. In particular embodiments, the antibody or a functional fragment thereof binds a TdT peptide comprising, consisting of, or consisting essentially of SEQ ID NO:3 or a variant thereof having at least 80, 85, 88, 90, or more % identity to the peptide.

The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986). Additionally, antigenic regions of proteins can also be predicted and identified using standard antigenicity and hydropathy plots.

The term “immunogenic sequence” means a molecule that includes an amino acid sequence of at least one epitope such that the molecule is capable of stimulating the production of antibodies in an appropriate host. The term “immunogenic composition” means a composition that comprises at least one immunogenic molecule (e.g., an antigen or carbohydrate).

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al., Front Immunol. 2013; 4: 302; 2013).

The term “light chain” includes a full-length light chain and fragments thereof having sufficient variable region sequence to confer binding specificity. A full-length light chain has a molecular weight of around 25,000 Daltons and includes a variable region domain (abbreviated herein as VL), and a constant region domain (abbreviated herein as CL). There are two classifications of light chains, identified as kappa (κ) and lambda (λ). The term “VL fragment” means a fragment of the light chain of a monoclonal antibody that includes all or part of the light chain variable region, including CDRs. A VL fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain has a molecular weight of around 50,000 Daltons and includes a variable region domain (abbreviated herein as VH), and three constant region domains (abbreviated herein as CH1, CH2, and CH3). The term “VH fragment” means a fragment of the heavy chain of a monoclonal antibody that includes all or part of the heavy chain variable region, including CDRs. A VH fragment can further include heavy chain constant region sequences. The number of heavy chain constant region domains will depend on the isotype. The VH domain is at the amino-terminus of the polypeptide, and the CH domains are at the carboxy-terminus, with the CH3 being closest to the —COOH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

In particular embodiments, the disclosure includes antibodies that bind a part of the TdT protein, including that bind a TdT peptide comprising, consisting of, or consisting essentially of SEQ ID NO:3. Although the antibody may be of any kind, in specific embodiments the antibody comprises a single chain variable fragment (scFv).

In various embodiments, the disclosure provides multiple scFvs that may be utilized alone or in any composition. In specific embodiments, the antibody is employed in a chimeric polypeptide of any kind, including any receptor and/or in any bi-specific or multi-specific antibody, including any bi-specific T-cell engager (BiTE). In specific embodiments, the antibodies comprise the following representative sequences:

Clone 1: TdT_B1 Polynucleotide Sequence
(SEQ ID NO: 4)
ATGGCCTTACCAGTGACCGCCTTGCTCCTGCCGCTGGCCTTGCTGCTC
CACGCCGCCAGGCCGGCTCAAGTTCAGCTGGTGCAGAGCGGAGCGGAG
GTGAAGAAGCCAGGGTCTTCCGTTAAGGTCTCTTGTAAGGCCTCAGGC
GGAACTTTCTCATCTTACGCTATTTCCTGGGTAAGGCAAGCACCAGGA
CAGGGATTAGAATGGATGGGGGGGATTATCCCCATTTTCGGTACCGCG
AACTACGCCCAAAAATTTCAAGGTCGGGTAACCATCACCGCCGATGAA
TCGACATCTACCGCCTACATGGAACTGAGCTCTCTTCGGAGCGAGGAC
ACCGCAGTCTATTACTGCGCGAGAGACGGGTATAGTGGTAGCTACTAC
TATTATTACGGCATGGACGTTTGGGGCCAGGGGACCTTAGTGACGGTG
AGCAGCGGAGGAGGCGGCGGGTCCCAGTCTGCTCTGACCCAACCTGCC
AGCGTCTCCGGCAGTCCAGGACAAAGTATCACCATCAGCTGCACTGGG
ACTTCCAGCGATGTGGGGGGCTACAATTACGTAAGTTGGTACCAGCAA
CACCCTGGGAAGGCCCCCAAGCTGATGATTTATGATGTCTCATACAGA
CCGAGCGGAGTGAGCCACCGTTTTAGTGGCTCCAAGTCCGGAAACACC
GCTTCACTAACAATCAGTGGCTTACAAGCAGAGGACGAAGCCGATTAT
TACTGCTCATCTTACACCAGTTCCAGTACGCTTGTGTTTGGTACTGGA
ACAAAGTTGACTGTGCTGGGC
Clone 1: TdT_B1 Polypeptide Sequence; signal
peptide-V_{H}-V_{L}
(SEQ ID NO: 5)
MALPVTALLLPLALLLHAARPAQVQLVQSGAEVKKPGSSVKVSCKAS
GGTFSSYAISWVRQAPGQGLEWMGGIIPIFGTANYAQKFQGRVTITA
DESTSTAYMELSSLRSEDTAVYYCARDGYSGSYYYYYGMDVWGQGTL
VTVSSGGGGGSQSALTQPASVSGSPGQSITISCTGTSSDVGGYNYVS
WYQQHPGKAPKLMIYDVSYRPSGVSHRFSGSKSGNTASLTISGLQAE
DEADYYCSSYTSSSTLVFGTGTKLTVL,
wherein the signal peptide is at the N-terminus
and lacks marking, the V_{H} domain is single
underlined, and the V_{L} domain is double
underlined.

In specific embodiments for B1, the V_{H} and V_{L} are as follows:

V_{H}:
(SEQ ID NO: 22)
AQVQLVQSGAEVKKPGSSVKVSCKASGGTFSSYAISWVRQAPGQGLEW
MGGIIPIFGTANYAQKFQGRVTITADESTSTAYMELSSLRSEDTAVYY
CARDGYSGSYYYYYGMDVWGQGTLVTVSS
V_{L}:
(SEQ ID NO: 25)
QSALTQPASVSGSPGQSITISCTGTSSDVGGYNYVSWYQQHPGKAPKL
MIYDVSYRPSGVSHRFSGSKSGNTASLTISGLQAEDEADYYCSSYTSS
STLVFGTGTKLTVL

In specific embodiments, the complementarity determining regions (CDRs) for B1 are as follows

HCDR1:
(SEQ ID NO: 28)
GGTFSSYA
HCDR2:
(SEQ ID NO: 29)
IIPIFGTA
HCDR3:
(SEQ ID NO: 30)
ARDGYSGSYYYYYGMDV
LCDR1:
(SEQ ID NO: 31)
SSDVGGYNY
LCDR2:
DVS
LCDR3:
(SEQ ID NO: 33)
SSYTSSSTLV
Clone 2: TdT_G7 Polynucleotide Sequence
(SEQ ID NO: 6)
ATGGCCTTACCAGTGACCGCCTTGCTCCTGCCGCTGGCCTTGCTGC
TCCACGCCGCCAGGCCGGCCCAAGTCCAGCTGGTTGAAAGCGGGGG
AGGAGTGGTTCAGCCCGGAAGGAGTTTGCGGTTGAGTTGTGCAGCA
TCAGGATTCACATTTTCTTCCTATGCAATGTCCTGGGTGCGTCAGG
CTCCCGGAAAGGGACTGGAATGGGTTAGCGCTATATCTGGCAGCGG
AGACTCTACATATTATGCTGATTCAGTCAAAGGCCGTTTCACCATC
AGCAGAGACAACAGTAAGAATACCCTCTACCTTCAGATGAACTCGC
TACGTGCTGAAGACACCGCCGTTTACTACTGTGCTAAAGATCTCGA
CTCCTCAAGTCCGGACGATGCTTTTGACATCTGGGGACAAGGCACC
ACTGTCACCGTCAGCAGCGGAGGCGCCGCGCTGGCCGAAGTGGCAG
CCGCGGTGGCTGACCCTATCGTTCTGACTCAGTCTCCTGGAAAGCT
GAGCCTCTCCCCAGGGGAGCGGGCCACTTTGTCCTGTGGCGCGTCG
CAGAGCGTATCATCCAATTACCTGGCATGGTACCAGCAGAAACCTG
GCCTGGCTCCTCGGCTTTTGATTTATGATGCTAGTATTAGGGCTAC
TGGCGTGCCTGATCGGTTCTCGGGATCAGGGAGCGCGACTGACTTC
ACTTTGACGATCAGCCGGCTGGACCCTGAGGATTTTGCGGTGTACT
ATTGCCACCAGTATTCATCTGCCCCCATGACTTTTGGACAGGGCAC
TAAGCTGGAGATTAAGAGG
Clone 2: TdT_G7 Polypeptide Sequence
(SEQ ID NO: 7)
MALPVTALLLPLALLLHAARPAQVQLVESGGGVVQPGRSLRLSCAA
SGFTFSSYAMSWVRQAPGKGLEWVSAISGSGDSTYYADSVKGRFTI
SRDNSKNTLYLQMNSLRAEDTAVYYCAKDLDSSSPDDAFDIWGQGT
TVTVSSGGAALAEVAAAVADPIVLTQSPGKLSLSPGERATLSCGAS
QSVSSNYLAWYQQKPGLAPRLLIYDASIRATGVPDRFSGSGSATDF
TLTISRLDPEDFAVYYCHQYSSAPMTFGQGTKLEIKR,
wherein the signal peptide is at the
N-terminus and lacks marking, the V_{H} domain
is single underlined, and the V_{L} domain is
double underlined.

In specific embodiments for G7, the VH and VL are as follows:

V_{H}:
(SEQ ID NO: 23)
AQVQLVESGGGVVQPGRSLRLSCAASGFTFSSYAMSWVRQAPGKGL
EWVSAISGSGDSTYYADSVKGRFTISRDNSKNTLYLQMNSLRAEDT
AVYYCAKDLDSSSPDDAFDIWGQGTTVTVSS
V_{L}:
(SEQ ID NO: 26)
AALAEVAAAVADPIVLTQSPGKLSLSPGERATLSCGASQSVSSNYL
AWYQQKPGLAPRLLIYDASIRATGVPDRFSGSGSATDFTLTISRLD
PEDFAVYYCHQYSSAPMTFGQGTKLEIKR

In specific embodiments, the complementarity determining regions (CDRs) for G7 are as follows

HCDR1:
(SEQ ID NO: 34)
GFTFSSYA
HCDR2:
(SEQ ID NO: 35)
ISGSGDST
HCDR3:
(SEQ ID NO: 36)
AKDLDSSSPDDAFDI
LCDR1:
(SEQ ID NO: 37)
QSVSSNY
LCDR2:
DAS
LCDR3:
(SEQ ID NO: 39)
HQYSSAPMT
Clone 3: TdT_G9 Polynucleotide Sequence
(SEQ ID NO: 8)
ATGGCCTTACCAGTGACCGCCTTGCTCCTGCCGCTGGCCTTGCTGC
TCCACGCCGCCAGGCCGGCTCAAGTGCAGCTGGTCGAATCTGGAGC
TGGGACTAAGAAGCCTGGCGAGAGTCTGAAGATCTCTTGTAAGGCC
AGCGGCTACAACTTTGCTTCTTACTGGATCGGGTGGGTTAGACAGA
TGCCTGGAAAAGGACTTGAGTGGGTAGGGATCATTGATCCTAGCGA
TTCCGATACCAAATACAGCCCAAGCTTTGAAGGGCAAGTCACTATT
AGTGTGGACAAGTCTATCAGCACTGCTCACCTGCAGTGGAGTAGCC
TAGATGCGAGCGACACGGCCATGTACTACTGCGCGAGGAGTCTGGG
CAGCTACTATGGAGATTGGTATTTCGATCTCTGGGGGAGGGGCACA
CTGGTGACCGTGTCATCCGGAGGAGGCGGCGGGTCCAACTTTATGC
TGACCCAGCCACATTCAATGTCTGAAAGCCCTGGCAAAACCGTAAC
AATCTCGTGCACACGGTCAAGCGGGAGCATCGCCAACAACTATGTT
CAGTGGTATCAGCAGAGACCCGGGAGTTCCCCTACCACTGTCATCT
ACGATGACAATCAGAGACCATCCGGGGTGCCCGACCGCTTCAGCGG
CTCAATAGATAGCTCCAGCAACTCCGCCTCTTTAACAATCTCGGGC
CTGAAAACCGAGGACGAAGCTGATTATTACTGCCAGTCCTATGACT
CCAGCAACGTGATTTTTGGTGGAGGGACTAAACTGACCGTCCTCGG
A
Clone 3: TdT_G9 Polypeptide Sequence
(SEQ ID NO: 9)
MALPVTALLLPLALLLHAARPAQVQLVESGAGTKKPGESLKISCKA
SGYNFASYWIGWVRQMPGKGLEWVGIIDPSDSDTKYSPSFEGQVTI
SVDKSISTAHLQWSSLDASDTAMYYCARSLGSYYGDWYFDLWGRGT
LVTVSSGGGGGSNFMLTQPHSMSESPGKTVTISCTRSSGSIANNYV
QWYQQRPGSSPTTVIYDDNQRPSGVPDRFSGSIDSSSNSASLTISG
LKTEDEADYYCQSYDSSNVIFGGGTKLTVLG,
wherein the signal peptide is at the
N-terminus and lacks marking, the V_{H} domain is
single underlined, and the V_{L} domain is double
underlined.

In specific embodiments for G9, the V_{H} and V_{L} are as follows:

V_{H}:
(SEQ ID NO: 24)
AQVQLVESGAGTKKPGESLKISCKASGYNFASYWIGWVRQMPGKGL
EWVGIIDPSDSDTKYSPSFEGQVTISVDKSISTAHLQWSSLDASDT
AMYYCARSLGSYYGDWYFDLWGRGTLVTVSS
V_{L}:
(SEQ ID NO: 27)
NFMLTQPHSMSESPGKTVTISCTRSSGSIANNYVQWYQQRPGSSPT
TVIYDDNQRPSGVPDRFSGSIDSSSNSASLTISGLKTEDEADYYCQ
SYDSSNVIFGGGTKLTVLG

In specific embodiments, the complementarity determining regions (CDRs) for G9 are as follows

HCDR1:
(SEQ ID NO: 40)
GYNFASYW
HCDR2:
(SEQ ID NO: 41)
IDPSDSDT
HCDR3:
(SEQ ID NO: 42)
ARSLGSYYGDWYFDL
LCDR1:
(SEQ ID NO: 43)
SGSIANNY
LCDR2:
DDN
LCDR3:
(SEQ ID NO: 45)
QSYDSSNVI

Thus, in specific embodiments, any antibody utilized herein may comprise one of the following V_{H} sequences or one or more modifications thereof:

(1)
(SEQ ID NO: 22)
AQVQLVQSGAEVKKPGSSVKVSCKASGGTFSSYAISWVRQAPGQGL
EWMGGIIPIFGTANYAQKFQGRVTITADESTSTAYMELSSLRSEDT
AVYYCARDGYSGSYYYYYGMDVWGQGTLVTVSS;
(2)
(SEQ ID NO: 23)
AQVQLVESGGGVVQPGRSLRLSCAASGFTFSSYAMSWVRQAPGKGL
EWVSAISGSGDSTYYADSVKGRFTISRDNSKNTLYLQMNSLRAEDT
AVYYCAKDLDSSSPDDAFDIWGQGTTVTVSS;
or
(3)
(SEQ ID NO: 24)
AQVQLVESGAGTKKPGESLKISCKASGYNFASYWIGWVRQMPGKGL
EWVGIIDPSDSDTKYSPSFEGQVTISVDKSISTAHLQWSSLDASDT
AMYYCARSLGSYYGDWYFDLWGRGTLVTVSS.

In specific embodiments, the V_{H} domain comprises SEQ ID NO:22, SEQID NO:23, or SEQ ID NO:24, or the V_{H} domain is at least 70, 75, 80, 85, 90, 91, 92, 93, 94, 95, 96, 97, 98, or 99% identical in sequence to SEQ ID NO:22, SEQ ID NO:23, or SEQ ID NO:24. The V_{H} domain may have 1, 2, 3, 4, 5, 6, 7, 8, 9, 10 or more modifications compared to SEQ ID NO:22, SEQID NO:23, or SEQ ID NO:24, including an amino acid substitution, an inversion, a deletion, and so forth. The N-terminal and/or C-terminal sequences may be truncated by 1, 2, 3, 4, 5, or more amino acids compared to SEQ ID NO:22, SEQ ID NO:23, or SEQ ID NO:24. In specific embodiments, the V_{H} domain comprises at least 100, 105, 110, 115, 116, 117, 118, 119, 120, 121, 122, or all contiguous amino acids of SEQ ID NO:22, SEQID NO:23, or SEQ ID NO:24.

Thus, in specific embodiments, any antibody utilized herein may comprise one of the following V_{L} sequences, or one or more modifications thereof:

(1)
(SEQ ID NO: 25)
QSALTQPASVSGSPGQSITISCTGTSSDVGGYNYVSWYQQHPGKAP
KLMIYDVSYRPSGVSHRFSGSKSGNTASLTISGLQAEDEADYYCSS
YTSSSTLVFGTGTKLTVL;
(2)
(SEQ ID NO: 26)
AALAEVAAAVADPIVLTQSPGKLSLSPGERATLSCGASQSVSSNYL
AWYQQKPGLAPRLLIYDASIRATGVPDRFSGSGSATDFTLTISRLD
PEDFAVYYCHQYSSAPMTFGQGTKLEIKR;
or
(3)
(SEQ ID NO: 27)
NFMLTQPHSMSESPGKTVTISCTRSSGSIANNYVQWYQQRPGSSPT
TVIYDDNQRPSGVPDRFSGSIDSSSNSASLTISGLKTEDEADYYCQ
SYDSSNVIFGGGTKLTVLG.

In specific embodiments, the V_{L} domain comprises SEQ ID NO:25, SEQ ID NO:26, or SEQ ID NO:27, or the V_{L} domain is at least 70, 75, 80, 85, 90, 91, 92, 93, 94, 95, 96, 97, 98, or 99% identical in sequence to SEQ ID NO:25, SEQID NO:26, or SEQ ID NO:27. The V_{L} domain may have 1, 2, 3, 4, 5, 6, 7, 8, 9, 10 or more modifications compared to SEQ ID NO:25, SEQ ID NO:26, or SEQ ID NO:27, including an amino acid substitution, an inversion, a deletion, and so forth. The N-terminal and/or C-terminal sequences may be truncated by 1, 2, 3, 4, 5, or more amino acids compared to SEQ ID NO:25, SEQ ID NO:26, or SEQ ID NO:27. In specific embodiments, the V_{H} domain comprises at least 100, 105, 110, 115, 116, 117, 118, 119, 120, 121, 122, or all contiguous amino acids of SEQ ID NO:25, SEQ ID NO:26, or SEQ ID NO:27.

The scFv in various embodiments comprises any one or more of SEQ ID NOs:4-9, 22-24, or 25-27. The scFv in specific embodiments comprises sequence that is at least 80, 85, 90, 91, 92, 93, 94, 95, 96, 97, 98, 99, or greater % identity to any one or more of SEQ ID NOs: 4-9, 22-24, or 25-27.

Aspects of the disclosure relate to an antibody or antigen binding fragment comprising a heavy chain variable region and a light chain variable region, wherein the heavy chain variable region comprises a HCDR1, HCDR2, and HCDR3 from a heavy chain variable region of an antibody clone from B1, G7, or G9 and wherein the light chain variable region comprises a LCDR1, LCDR2, and LCDR3 from the light chain variable region of the same respective clone of B1, G7, or G9. Further aspects relate to an antibody or antigen binding fragment comprising a heavy chain variable region and a light chain variable region, wherein the heavy chain variable region comprises a HCDR1, HCDR2, and HCDR3 having or having at least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a HCDR1, HCDR2, and HCDR3 from a heavy chain variable region of an antibody clone of B1, G7, or G9, and wherein the light chain variable region comprises a LCDR1, LCDR2, and LCDR3 having or having at least least 80% sequence identity or having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% (or any derivable range therein) sequence identity with a LCDR1, LCDR2, and LCDR3 from the light chain variable region of the same respective clone of B1, G7, or G9.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:28-30, respectively and the LCDR1, LCDR2, and LCDR3 comprises an amino acid sequence of SEQ ID NOS:31, 33 or “DVS”, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:34-36, respectively and the LCDR1, LCDR2, and LCDR3 comprises an amino acid sequence of SEQ ID NOS:37, 39 or “DAS”, respectively.

Aspects of the disclosure relate to an antibody, antigen binding fragment, or polypeptide comprising a heavy chain variable region having a HCDR1, HCDR2, and HCDR3, and a light chain variable region having a LCDR1, LCDR2, and LCDR3, wherein the HCDR1, HCDR2, and HCDR3 comprises an amino acid sequence of SEQ ID NOS:40-42, respectively and the LCDR1, LCDR2, and LCDR3 comprises an amino acid sequence of SEQ ID NOS:43, 45 or “DDN”, respectively.

Polynucleotides encoding the antibody or parts thereof, the antibody proteins, vectors that comprise the polynucleotide, vectors that encode the antibody proteins, and cells that encompass any of these agents are all encompassed herein. In particular embodiments, the antibody is utilized in a chimeric protein of any kind. Polynucleotides encoding the chimeric protein, the chimeric protein, vectors that comprise the polynucleotide that encodes the chimeric protein, and cells that encompass any of these agents are all encompassed herein. The chimeric protein may or may not be a chimeric receptor, a chimeric antibody, multi-specific antibodies, labeled antibodies, antibody conjugates, and so forth.

### A. TdT Peptide Antibody-Based Compositions

The present disclosure concerns at least treatment and/or detection methods using a specific antibody that targets one or more TdT peptides and compositions that utilize them. In various embodiments, a particular antibody is employed in one or more different compositions for targeting cancer cells that express a TdT peptide, and in specific embodiments the peptide comprises ALYDKTKRI (SEQ ID NO:3). In some embodiments, one of the compositions that utilizes the antibody is cell-based, is expressed in a non-natural receptor on the surface of the cell, and targets the TdT. In some embodiments, one of the compositions that utilizes the antibody is soluble and targets the TdT peptide outside of a cell (although it may be delivered to an individual in the form of a cell following which the soluble protein comprising the antibody is secreted from the cell).

The present disclosure also concerns methods of utilizing any TdT peptide antibody-based composition for recognition of the TdT peptide for any reason. Such methods may include detection of TdT peptide in a sample in need of processing. The sample may derive from an individual in need of determination of presence of the TdT peptide for any reason. In specific embodiments, the TdT peptide antibody-based composition is used for the detection of cancer in a sample, such as detetion of cancer cells in the sample that express the TdT peptide, including in at least some cases one association with an HLA-A2 complex. The TdT peptide may be associated with any HLA antigen, including HLA-A, HLA-B, HLA-C, HLA-E, HLA-F or HLA-G.

In particular embodiments, the disclosure includes antibodies that bind a part of the TdT protein, including that bind a TdT peptide comprising, consisting of, or consisting essentially of SEQ ID NO:3, or a variant thereof. Although the antibody may be of any kind, in specific embodiments the antibody comprises a single chain variable fragment (scFv).

In various embodiments, the disclosure provides multiple scFvs that may be utilized alone or in any chimeric polypeptide, including any receptor and/or in any bi-specific or multi-specific antibody, including any BiTE, or in any conjugate. In specific embodiments, the antibodies comprise sequences disclosed herein.

The scFv in various embodiments comprises any one or more of SEQ ID NOs:4-9 or 22-27. The scFv in specific embodiments comprises sequence that is at least 80, 85, 90, 91, 92, 93, 94, 95, 96, 97, 98, 99, or greater % identity to any one or more of SEQ ID NOs:4-9 or 22-27. In specific embodiments, the scFv comprises a complementarity-determining region(s) that comprises sequence identified by standard means in the art.

Polynucleotides encoding the antibody or parts thereof, the antibody proteins, vectors that comprise the polynucleotide, and vectors that encode the antibody proteins are all encompassed herein. In particular embodiments, the antibody is utilized in a chimeric protein of any kind. In a particular embodiment, the chimeric protein is a receptor, including a receptor that binds a TdT peptide, such as a receptor that comprises an antibody that binds a TdT peptide. In a particular embodiment, the chimeric receptor is a chimeric T cell receptor (TCR) or a chimeric antigen receptor. Polynucleotides that encode the chimeric TCR comprising the antibody, the chimeric TCR protein, vectors that comprise the polynucleotide, and vectors that encode the chimeric TCR protein are all encompassed herein. The antibody portion of the chimeric TCR may be configured in a variety of ways with respect to the TCR α and β chains. Any vector may be utilized, including viral vectors and non-viral vectors. The viral vector may be an adenoviral vector, adeno-associated viral vector, retroviral vector, or lentiviral vector. A non-viral vector may be a plasmid or transposon.

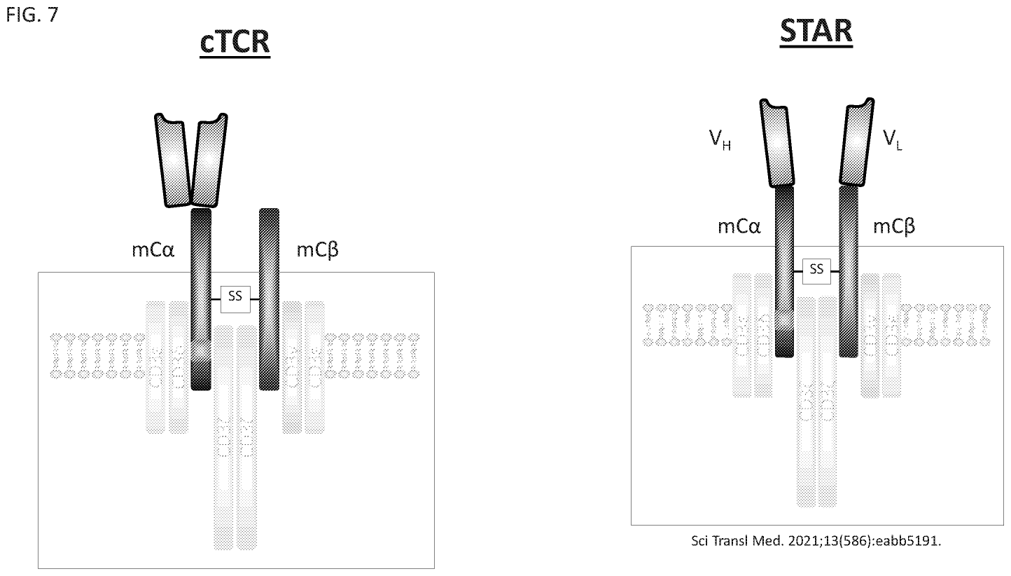

In one embodiment, the chimeric receptor is a synthetic T cell receptor (TCR) and antigen receptor (STAR) (Liu et al., Sci Transl Med. 2021; 13(586):eabb5191) that comprise antigen-recognition domains of antibody with constant regions of a TCR molecule (FIG. 7). In various embodiments, the STAR receptor is a double-chain TCRab-based receptor with variable regions of immunoglobulin heavy and light chains (V_{H} and V_{L}) fused to TCR-Ca and TCR-Cb domains. In specific embodiments, a STAR receptor comprises a fusion of V_{H} and V_{L} to TCR α and β chain constant regions, respectively. Although one representative example of a STAR receptor that comprises the B1 antibody referred to above and comprises two separate chains (the B1 STAR), any antibody that binds TdT peptide may be utilized likewise with any STAR configuration.

B1_STAR (2 separate chains):
B1_STAR VH-TCRA Polynucleotide (the V_{H} domain
of the B1 antibody linked to the TCR alpha
chain)
(SEQ ID NO: 10)
ATGGCCTTACCAGTGACCGCCTTGCTCCTGCCGCTGGCCTTGCTGC
TCCACGCCGCCAGGCCGGCTCAAGTTCAGCTGGTGCAGAGCGGAGC
GGAGGTGAAGAAGCCAGGGTCTTCCGTTAAGGTCTCTTGTAAGGCC
TCAGGCGGAACTTTCTCATCTTACGCTATTTCCTGGGTAAGGCAAG
CACCAGGACAGGGATTAGAATGGATGGGGGGGATTATCCCCATTTT
CGGTACCGCGAACTACGCCCAAAAATTTCAAGGTCGGGTAACCATC
ACCGCCGATGAATCGACATCTACCGCCTACATGGAACTGAGCTCTC
TTCGGAGCGAGGACACCGCAGTCTATTACTGCGCGAGAGACGGGTA
TAGTGGTAGCTACTACTATTATTACGGCATGGACGTTTGGGGCCAG
GGGACCTTAGTGACGGTGAGCAGCGGATCCCCATACATTCAAAATC
CAGAACCTGCGGTATACCAATTGAAAGACCCAAGAAGCCAGGATTC
CACGCTGTGTCTGTTTACAGACTTCGACTCACAGATCAATGTTCCC
AAGACAATGGAAAGCGGGACGTTCATCACCGATAAATGCGTTCTGG
ACATGAAGGCTATGGATTCTAAAAGTAATGGTGCTATAGCCTGGAG
TAACCAGACCTCATTTACTTGCCAAGACATTTTCAAGGAGACCAAT
GCTACATATCCATCCTCCGACGTCCCTTGTGACGCCACTCTGACCG
AGAAATCTTTTGAGACCGATATGAACCTGAACTTTCAGAACCTACT
GGTTATCGTTCTGAGAATACTGTTGCTCAAAGTCGCCGGATTCAAC
CTTCTCATGACTCTGAGACTGTGGAGTAGC
B1_STAR VH-TCRA Polypeptide (the V_{H} domain of
the B1 antibody linked to the TCR alpha chain)
(SEQ ID NO: 11)
MALPVTALLLPLALLLHAARPAQVQLVQSGAEVKKPGSSVKVSCKA
SGGTFSSYAISWVRQAPGQGLEWMGGIIPIFGTANYAQKFQGRVTI
TADESTSTAYMELSSLRSEDTAVYYCARDGYSGSYYYYYGMDVWGQ
GTLVTVSSGSPYIQNPEPAVYQLKDPRSQDSTLCLFTDFDSQINVP
KTMESGTFITDKCVLDMKAMDSKSNGAIAWSNQTSFTCQDIFKETN
ATYPSSDVPCDATLTEKSFETDMNLNFQNLLVIVLRILLLKVAGFN
LLMTLRLWSS
B1_STAR VL-TCRB Polynucleotide (the V_{L} domain
of the B1 antibody linked to the TCR beta
chain)
(SEQ ID NO: 12)
ATGGAGACAGACACACTCCTGctatgggtgctgctgctctGGGTTC
CAGGTTCCACAGGTCAGTCTGCTCTGACCCAACCTGCCAGCGTCTC
CGGCAGTCCAGGACAAAGTATCACCATCAGCTGCACTGGGACTTCC
AGCGATGTGGGGGGCTACAATTACGTAAGTTGGTACCAGCAACACC
CTGGGAAGGCCCCCAAGCTGATGATTTATGATGTCTCATACAGACC
GAGCGGAGTGAGCCACCGTTTTAGTGGCTCCAAGTCCGGAAACACC
GCTTCACTAACAATCAGTGGCTTACAAGCAGAGGACGAAGCCGATT
ATTACTGCTCATCTTACACCAGTTCCAGTACGCTTGTGTTTGGTAC
TGGAACAAAGTTGACTGTGCTGGGCACGCGTGAGGACTTGAGGAAT
GTGACGCCACCGAAGGTCTCTTTGTTTGAGCCGTCTAAGGCAGAAA
TTGCTAATAAACAAAAGGCCACCCTAGTGTGTCTCGCGAGGGGATT
CTTTCCTGATCACGTTGAGTTGTCCTGGTGGGTGAATGGTAAGGAG
GTTCATAGCGGTGTCTGCACCGACCCCCAGGCATACAAGGAGTCGA
ACTACTCCTACTGCCTGTCATCTAGACTCCGAGTCAGTGCCACGTT
CTGGCATAACCCCCGTAACCATTTTAGGTGCCAGGTGCAATTCCAC
GGGCTTAGTGAAGAGGACAAGTGGCCAGAGGGGTCCCCAAAGCCGG
TGACCCAAAATATCTCTGCGGAAGCTTGGGGGCGCGCCGATTGTGG
GATTACCAGCGCGAGCTATCAGCAGGGAGTCCTGAGTGCAACGATA
CTCTACGAGATATTACTGGGAAAGGCTACATTATATGCAGTGTTGG
TGAGCACTCTGGTCGTCATGGCAATGGTTAAGAGAAAGAATAGCTA
A
B1_STAR VL-TCRB Polypeptide (the V_{L} domain of
the B1 antibody linked to the TCR beta chain)
(SEQ ID NO: 13)
METDTLLLWVLLLWVPGSTGQSALTQPASVSGSPGQSITISCTGTS
SDVGGYNYVSWYQQHPGKAPKLMIYDVSYRPSGVSHRFSGSKSGNT
ASLTISGLQAEDEADYYCSSYTSSSTLVFGTGTKLTVLGTREDLRN
VTPPKVSLFEPSKAEIANKQKATLVCLARGFFPDHVELSWWVNGKE
VHSGVCTDPQAYKESNYSYCLSSRLRVSATFWHNPRNHFRCQVQFH
GLSEEDKWPEGSPKPVTQNISAEAWGRADCGITSASYQQGVLSATI
LYEILLGKATLYAVLVSTLVVMAMVKRKNS

In one embodiment, the chimeric receptor is a chimeric T-cell receptor (cTCR). In such cases, an scFv is fused to the TCRa and/or the TCRb chains. In a specific embodiment, both of the VH and VL domains are linked either to a TCR alpha chain or to a TCR beta chain. Although one representative example of a cTCR comprises the B1 antibody referred to above linked to TCR α or β chains (B1 cTCR), any antibody that binds TdT peptide may be utilized likewise with any configuration of TCR ab chains. In cases wherein both the V_{L} and V_{H} domains of an antibody are linked to the TCR alpha chain, the TCR beta chain would be considered “empty” (e.g., lacking the variable domain sequences). In cases wherein the V_{L} and V_{H} domains of an antibody are linked to the TCR beta chain, the TCR alpha chain would be considered “empty” (e.g., lacking the variable domain sequences).

B1.cTCR:
B1_cTCR VHVL-TCRA Polynucleotide (the V_{L} and
V_{H} domains of the B1 antibody linked to the
TCR alpha chain)
(SEQ ID NO: 14)
ATGGCCTTACCAGTGACCGCCTTGCTCCTGCCGCTGGCCTTGCTGC
TCCACGCCGCCAGGCCGGCTCAAGTTCAGCTGGTGCAGAGCGGAGC
GGAGGTGAAGAAGCCAGGGTCTTCCGTTAAGGTCTCTTGTAAGGCC
TCAGGCGGAACTTTCTCATCTTACGCTATTTCCTGGGTAAGGCAAG
CACCAGGACAGGGATTAGAATGGATGGGGGGGATTATCCCCATTTT
CGGTACCGCGAACTACGCCCAAAAATTTCAAGGTCGGGTAACCATC
ACCGCCGATGAATCGACATCTACCGCCTACATGGAACTGAGCTCTC
TTCGGAGCGAGGACACCGCAGTCTATTACTGCGCGAGAGACGGGTA
TAGTGGTAGCTACTACTATTATTACGGCATGGACGTTTGGGGCCAG
GGGACCTTAGTGACGGTGAGCAGCGGAGGAGGCGGCGGGTCCCAGT
CTGCTCTGACCCAACCTGCCAGCGTCTCCGGCAGTCCAGGACAAAG
TATCACCATCAGCTGCACTGGGACTTCCAGCGATGTGGGGGGCTAC
AATTACGTAAGTTGGTACCAGCAACACCCTGGGAAGGCCCCCAAGC
TGATGATTTATGATGTCTCATACAGACCGAGCGGAGTGAGCCACCG
TTTTAGTGGCTCCAAGTCCGGAAACACCGCTTCACTAACAATCAGT
GGCTTACAAGCAGAGGACGAAGCCGATTATTACTGCTCATCTTACA
CCAGTTCCAGTACGCTTGTGTTTGGTACTGGAACAAAGTTGACTGT
GCTGGGCGGATCCCCATACATTCAAAATCCAGAACCTGCGGTATAC
CAATTGAAAGACCCAAGAAGCCAGGATTCCACGCTGTGTCTGTTTA
CAGACTTCGACTCACAGATCAATGTTCCCAAGACAATGGAAAGCGG
GACGTTCATCACCGATAAATGCGTTCTGGACATGAAGGCTATGGAT
TCTAAAAGTAATGGTGCTATAGCCTGGAGTAACCAGACCTCATTTA
CTTGCCAAGACATTTTCAAGGAGACCAATGCTACATATCCATCCTC
CGACGTCCCTTGTGACGCCACTCTGACCGAGAAATCTTTTGAGACC
GATATGAACCTGAACTTTCAGAACCTACTGGTTATCGTTCTGAGAA
TACTGTTGCTCAAAGTCGCCGGATTCAACCTTCTCATGACTCTGAG
ACTGTGGAGTAGC
B1_cTCR VHVL-TCRA Polypeptide (the V_{L} and V_{H}
domains of the B1 antibody linked to the TCR
alpha chain)
(SEQ ID NO: 15)
MALPVTALLLPLALLLHAARPAQVQLVQSGAEVKKPGSSVKVSCKA
SGGTFSSYAISWVRQAPGQGLEWMGGIIPIFGTANYAQKFQGRVTI
TADESTSTAYMELSSLRSEDTAVYYCARDGYSGSYYYYYGMDVWGQ
GTLVTVSSGGGGGSQSALTQPASVSGSPGQSITISCTGTSSDVGGY
NYVSWYQQHPGKAPKLMIYDVSYRPSGVSHRFSGSKSGNTASLTIS
GLQAEDEADYYCSSYTSSSTLVFGTGTKLTVLGGSPYIQNPEPAVY
QLKDPRSQDSTLCLFTDFDSQINVPKTMESGTFITDKCVLDMKAMD
SKSNGAIAWSNQTSFTCQDIFKETNATYPSSDVPCDATLTEKSFET
DMNLNFQNLLVIVLRILLLKVAGFNLLMTLRLWSS
B1_cTCR VHVL-TCRB Polynucleotide (the V_{L} and
V_{H} domains of the B1 antibody linked to the
TCR beta chain)
(SEQ ID NO: 16)
ATGGCCTTACCAGTGACCGCCTTGCTCCTGCCGCTGGCCTTGCTGC
TCCACGCCGCCAGGCCGGCTCAAGTTCAGCTGGTGCAGAGCGGAGC
GGAGGTGAAGAAGCCAGGGTCTTCCGTTAAGGTCTCTTGTAAGGCC
TCAGGCGGAACTTTCTCATCTTACGCTATTTCCTGGGTAAGGCAAG
CACCAGGACAGGGATTAGAATGGATGGGGGGGATTATCCCCATTTT
CGGTACCGCGAACTACGCCCAAAAATTTCAAGGTCGGGTAACCATC
ACCGCCGATGAATCGACATCTACCGCCTACATGGAACTGAGCTCTC
TTCGGAGCGAGGACACCGCAGTCTATTACTGCGCGAGAGACGGGTA
TAGTGGTAGCTACTACTATTATTACGGCATGGACGTTTGGGGCCAG
GGGACCTTAGTGACGGTGAGCAGCGGAGGAGGCGGCGGGTCCCAGT
CTGCTCTGACCCAACCTGCCAGCGTCTCCGGCAGTCCAGGACAAAG
TATCACCATCAGCTGCACTGGGACTTCCAGCGATGTGGGGGGCTAC
AATTACGTAAGTTGGTACCAGCAACACCCTGGGAAGGCCCCCAAGC
TGATGATTTATGATGTCTCATACAGACCGAGCGGAGTGAGCCACCG
TTTTAGTGGCTCCAAGTCCGGAAACACCGCTTCACTAACAATCAGT
GGCTTACAAGCAGAGGACGAAGCCGATTATTACTGCTCATCTTACA
CCAGTTCCAGTACGCTTGTGTTTGGTACTGGAACAAAGTTGACTGT
GCTGGGCGGATCCACGCGTGAGGACTTGAGGAATGTGACGCCACCG
AAGGTCTCTTTGTTTGAGCCGTCTAAGGCAGAAATTGCTAATAAAC
AAAAGGCCACCCTAGTGTGTCTCGCGAGGGGATTCTTTCCTGATCA
CGTTGAGTTGTCCTGGTGGGTGAATGGTAAGGAGGTTCATAGCGGT
GTCTGCACCGACCCCCAGGCATACAAGGAGTCGAACTACTCCTACT
GCCTGTCATCTAGACTCCGAGTCAGTGCCACGTTCTGGCATAACCC
CCGTAACCATTTTAGGTGCCAGGTGCAATTCCACGGGCTTAGTGAA
GAGGACAAGTGGCCAGAGGGGTCCCCAAAGCCGGTGACCCAAAATA
TCTCTGCGGAAGCTTGGGGGCGCGCCGATTGTGGGATTACCAGCGC
GAGCTATCAGCAGGGAGTCCTGAGTGCAACGATACTCTACGAGATA
TTACTGGGAAAGGCTACATTATATGCAGTGTTGGTGAGCACTCTGG
TCGTCATGGCAATGGTTAAGAGAAAGAATAGCTAA
B1_cTCR VHVL-TCRB Polypeptide (the V_{L} and V_{H}
domains of the B1 antibody linked to the TCR
beta chain)
(SEQ ID NO: 17)
MALPVTALLLPLALLLHAARPAQVQLVQSGAEVKKPGSSVKVSCKA
SGGTFSSYAISWVRQAPGQGLEWMGGIIPIFGTANYAQKFQGRVTI
TADESTSTAYMELSSLRSEDTAVYYCARDGYSGSYYYYYGMDVWGQ
GTLVTVSSGGGGGSQSALTQPASVSGSPGQSITISCTGTSSDVGGY
NYVSWYQQHPGKAPKLMIYDVSYRPSGVSHRFSGSKSGNTASLTIS
GLQAEDEADYYCSSYTSSSTLVFGTGTKLTVLGGSTREDLRNVTPP
KVSLFEPSKAEIANKQKATLVCLARGFFPDHVELSWWVNGKEVHSG
VCTDPQAYKESNYSYCLSSRLRVSATFWHNPRNHFRCQVQFHGLSE
EDKWPEGSPKPVTQNISAEAWGRADCGITSASYQQGVLSATILYEI
LLGKATLYAVLVSTLVVMAMVKRKNS.

One example of a TCR beta chain considered “empty” (lacking any antibody material) is as follows and may be combined in a cell with a TCR alpha chain itself linked to V_{H} and V_{L} domains:

TCRb_empty Polynucleotide
(SEQ ID NO: 20)
ATGGAGACAGACACACTCCTGctatgggtgctgctgctctgggttC
CAGGTTCCACAGGTGGCGGCGGTGGGTCAGAACAAAAATTGATTTC
AGAGGAAGATCTCGGAGGGGGAGGGTCAACGCGTGAGGACTTGAGG
AATGTGACGCCACCGAAGGTCTCTTTGTTTGAGCCGTCTAAGGCAG
AAATTGCTAATAAACAAAAGGCCACCCTAGTGTGTCTCGCGAGGGG
ATTCTTTCCTGATCACGTTGAGTTGTCCTGGTGGGTGAATGGTAAG
GAGGTTCATAGCGGTGTCTGCACCGACCCCCAGGCATACAAGGAGT
CGAACTACTCCTACTGCCTGTCATCTAGACTCCGAGTCAGTGCCAC
GTTCTGGCATAACCCCCGTAACCATTTTAGGTGCCAGGTGCAATTC
CACGGGCTTAGTGAAGAGGACAAGTGGCCAGAGGGGTCCCCAAAGC
CGGTGACCCAAAATATCTCTGCGGAAGCTTGGGGGCGCGCCGATTG
TGGGATTACCAGCGCGAGCTATCAGCAGGGAGTCCTGAGTGCAACG
ATACTCTACGAGATATTACTGGGAAAGGCTACATTATATGCAGTGT
TGGTGAGCACTCTGGTCGTCATGGCAATGGTTAAGAGAAAGAATAG
CTAA
TCRb_empty Polypeptide
(SEQ ID NO: 21)
METDTLLLWVLLLWVPGSTGGGGGSEQKLISEEDLGGGGSTREDLR
NVTPPKVSLFEPSKAEIANKQKATLVCLARGFFPDHVELSWWVNGK
EVHSGVCTDPQAYKESNYSYCLSSRLRVSATFWHNPRNHFRCQVQF
HGLSEEDKWPEGSPKPVTQNISAEAWGRADCGITSASYQQGVLSAT
ILYEILLGKATLYAVLVSTLVVMAMVKRKNS

Representative sequences of TCR alpha chains that are “empty” (lacking any antibody material) are known in the art.

In one embodiment, there are methods and compositions that utilize a bi-specific T cell engager of a specific sequence that comprises any antibody that binds a TdT peptide. Although a specific example of an engager utilizes the B1 antibody clone referred to above, in other embodiments any TdT-specific antibody is employed in a BiTE.

TdT B1 BiTE Polynucleotide (B1 antibody linked
to anti-CD3 antibody)
(SEQ ID NO: 18)
ATGGATTGGATCTGGCGCATCCTGTTTCTCGTGGGAGCCGCCACAG
GCGCCCATTCTGCTCAAGTTCAGCTGGTGCAGAGCGGAGCGGAGGT
GAAGAAGCCAGGGTCTTCCGTTAAGGTCTCTTGTAAGGCCTCAGGC
GGAACTTTCTCATCTTACGCTATTTCCTGGGTAAGGCAAGCACCAG
GACAGGGATTAGAATGGATGGGGGGGATTATCCCCATTTTCGGTAC
CGCGAACTACGCCCAAAAATTTCAAGGTCGGGTAACCATCACCGCC
GATGAATCGACATCTACCGCCTACATGGAACTGAGCTCTCTTCGGA
GCGAGGACACCGCAGTCTATTACTGCGCGAGAGACGGGTATAGTGG
TAGCTACTACTATTATTACGGCATGGACGTTTGGGGCCAGGGGACC
TTAGTGACGGTGAGCAGCGGAGGAGGCGGCGGGTCCCAGTCTGCTC
TGACCCAACCTGCCAGCGTCTCCGGCAGTCCAGGACAAAGTATCAC
CATCAGCTGCACTGGGACTTCCAGCGATGTGGGGGGCTACAATTAC
GTAAGTTGGTACCAGCAACACCCTGGGAAGGCCCCCAAGCTGATGA
TTTATGATGTCTCATACAGACCGAGCGGAGTGAGCCACCGTTTTAG
TGGCTCCAAGTCCGGAAACACCGCTTCACTAACAATCAGTGGCTTA
CAAGCAGAGGACGAAGCCGATTATTACTGCTCATCTTACACCAGTT
CCAGTACGCTTGTGTTTGGTACTGGAACAAAGTTGACTGTGCTGGG
CTCCGGAGGTGGTGGATCCGATATCAAACTGCAGCAGTCAGGGGCT
GAACTGGCAAGACCTGGGGCCTCAGTGAAGATGTCCTGCAAGACTT
CTGGCTACACCTTTACTAGGTACACGATGCACTGGGTAAAACAGAG
GCCTGGACAGGGTCTGGAATGGATTGGATACATTAATCCTAGCCGT
GGTTATACTAATTACAATCAGAAGTTCAAGGACAAGGCCACATTGA
CTACAGACAAATCCTCCAGCACAGCCTACATGCAACTGAGCAGCCT
GACATCTGAGGACTCTGCAGTCTATTACTGTGCAAGATATTATGAT
GATCATTACTGCCTTGACTACTGGGGCCAAGGCACCACTCTCACAG
TCTCCTCAGGTGGTGGTGGTTCTGGCGGCGGCGGCTCCGGTGGTGG
TGGTTCTGACATTCAGCTGACCCAGTCTCCAGCAATCATGTCTGCA
TCTCCAGGGGAGAAGGTCACCATGACCTGCAGAGCCAGTTCAAGTG
TAAGTTACATGAACTGGTACCAGCAGAAGTCAGGCACCTCCCCCAA
AAGATGGATTTATGACACATCCAAAGTGGCTTCTGGAGTCCCTTAT
CGCTTCAGTGGCAGTGGGTCTGGGACCTCATACTCTCTCACAATCA
GCAGCATGGAGGCTGAAGATGCTGCCACTTATTACTGCCAACAGTG
GAGTAGTAACCCGCTCACGTTCGGTGCTGGGACCAAGCTGGAGCTG
AAATCCTAA
TdT B1 BiTE Polypeptide (B1 antibody linked to
anti-CD3 antibody)
(SEQ ID NO: 19)
MDWIWRILFLVGAATGAHSAQVQLVQSGAEVKKPGSSVKVSCKASG
GTFSSYAISWVRQAPGQGLEWMGGIIPIFGTANYAQKFQGRVTITA
DESTSTAYMELSSLRSEDTAVYYCARDGYSGSYYYYYGMDVWGQGT
LVTVSSGGGGGSQSALTQPASVSGSPGQSITISCTGTSSDVGGYNY
VSWYQQHPGKAPKLMIYDVSYRPSGVSHRFSGSKSGNTASLTISGL
QAEDEADYYCSSYTSSSTLVFGTGTKLTVLGSGGGGSDIKLQQSGA
ELARPGASVKMSCKTSGYTFTRYTMHWVKQRPGQGLEWIGYINPSR
GYTNYNQKFKDKATLTTDKSSSTAYMQLSSLTSEDSAVYYCARYYD
DHYCLDYWGQGTTLTVSSGGGGSGGGGSGGGGSDIQLTQSPAIMSA
SPGEKVTMTCRASSSVSYMNWYQQKSGTSPKRWIYDTSKVASGVPY
RFSGSGSGTSYSLTISSMEAEDAATYYCQQWSSNPLTFGAGTKLEL
KS

In various embodiments, any polynucleotide that comprises sequence that encodes chimeric receptor protein (chimeric TCR or STAR or chimeric antigen receptor) may also encode one or more other gene products. In particular embodiments, the polynucleotide that encodes the chimeric receptor protein may also encode another gene product that also uses an antibody, including the anti-TdT peptide antibody. In specific embodiments, the polynucleotide that encodes the chimeric receptor protein further comprises sequence that encodes a bispecific T-cell engager (BiTE), including one that comprises the anti-TdT peptide antibody. In specific cases, the the polynucleotide that encodes the TCR protein is a different polynucleotide that encodes the BiTE that comprises an antibody specific for TdT or a TdT peptide.

In particular embodiments, the chimeric receptor that is utilized may include a chimeric antigen receptor (CAR). The CAR may be first generation, second generation, third generation, and so on. In specific embodiments, the antibody portion of the CAR targets the TdT peptide which may or may not be associated with an HLA. The hinge, transmembrane domain, CD3zeta (or a domain with a similar function), and optionally one or more costimulatory domains may be of any suitable kind.

In specific embodiments, the disclosure concerns TdT-specific cytotoxic receptors and methods of their use. This disclosure encompasses novel reagents that recognize and eliminate cancer cells of any kind (including hematological cancers, such as acute leukemia cells) without producing substantial damage to normal cells. The disclosure includes a T-cell-based (chimeric T-cell receptor (TCR)) and/or a recombinant protein-based (bispecific T-cell engager) reagent to target a specific TdT peptide, including one presented on the cell surface, such as in the context of HLA-A2. Potent activity of TdT-redirected T-cells in preclinical models of human acute leukemia is demonstrated herein.

Enhancing sensitivity of tumor antigen targeting by chimeric receptors is encompassed herein. Active development of CAR- and TCR-based immune cell therapies and their clinical evaluation have prompted investigation of the mechanisms regulating tumor-specific receptor sensitivity and efficacy. While most CARs target antigens that are abundantly expressed on tumor cells, it is important to understand the limits of their antigen sensitivity and devise new strategies to overcome low responsiveness to tumors with reduced expression of the target antigen. Prior studies have suggested CARs have a significantly reduced sensitivity compared to TCRs and are prone to “ignoring” tumor variants with decreased surface density of the antigen (Watanabe et al., 2015; Gudipati et al., 2020). To overcome this limitation, the inventors combined high TCR sensitivity with the versatility of CAR-based targeting and developed a highly sensitive chimeric TCR and an antibody-derived antigen recognition moiety.

Embodiments of the disclosure allow for redirecting of both CD4+ and CD8+ T-cells against an MHC class I-presented tumor antigen. T-cells expressing naturally-occurring TdT-specific TCRs are deleted during thymic selection, thus precluding selection or expansion of natural TdT-specific T cells in patients in need of them, such as with leukemia. To overcome this limitation, the inventors identified an antibody binder that specifically recognizes a dominant TdT peptide in the context of HLA-A02, the most common MHC class I allele in the Western world. Because this binding does not require the CD8 coreceptor, expression of a TdT-specific cTCR effectively redirects both CD8+ and CD4+ T-cells to elicit effector functions against TdT+ leukemia, unlike conventional MHC I-specific TCRs that only activate CD8+ T-cells. Based on the current preclinical and clinical evidence, CD4+ T-cell activation improves TdT-specific T-cell responses and overall anti-leukemic activity, in specific embodiments.

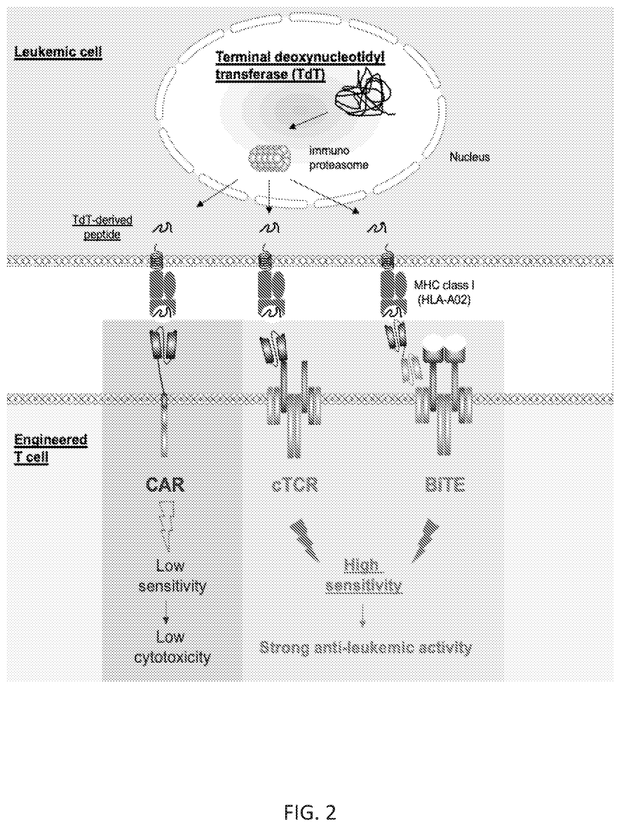

As demonstrated elsewhere herein, T cells expressing a TdT-specific cTCR have more potent anti-leukemic activity compared to those expressing an analogous TdT-specific chimeric antigen receptor (CAR). These results indicate that in various embodiments optimal recognition of rare TdT/HLA complexes requires highly sensitive TCR signaling. As shown herein, unmodified T-cells can be effectively redirected against TdT+ leukemia in the presence of soluble TdT-specific T-cell engagers that similarly activates TCR signaling in bystander, nonmodified T-cells. These results provide the scientific basis for developing both cellular and acellular strategies to target TdT using cTCR-transgenic T-cells or recombinant bispecific engagers, respectively. Compared to autologous engineered T-cells, bispecific engagers offer off-the-shelf availability and reduced manufacturing cost, and their application in the course of treatment, including early in the course of treatment, may improve antitumor activity of standard of care therapy, especially in patients with high-risk T-ALL and AML (FIG. 2). Thus, in specific embodiments of the disclosure, there are methods and compositions that employ adoptive T-cell therapy and bispecific T-cell engagers for the same individual.

In various embodiments, methods and compositions concern a recombinant T-cell receptor (TCR) comprising a single-chain fragment variable (scFv) specific for a terminal deoxynucleotidyl transferase (TdT) or a TdT peptide. Any embodiments associated with the receptor may be utilized, including the non-natural TCR polypeptide, recombinant polynucleotides that encode the TCR polypeptide, cells (including those isolated from nature, commercially obtained, obtained from a donor or an individual to be treated with them, etc.) that comprise the TCR polypeptide or polynucleotides that encode the TCR polypeptide, vector polynucleotides of any kind that express the TCR polypeptide, cells that harbor one or more recombinant polynucleotides, and so forth. In particular embodiments of the recombinant TCR, the TCR comprises a binder, such as an antibody, that targets a TdT peptide, including one that may be associated with HLA-A02 on cell surfaces (including cancer cell surfaces, as opposed to non-cancerous cells).

The TCR is non-natural for having been engineered by the hand of man to include an antibody that binds a TdT peptide and in some embodiments structurally comprises a disulfide-linked membrane-anchored heterodimeric protein comprising α chain and β chain. In specific embodiments, a TCR α chain and/or TCR β chain in the recombinant TCR have one or more modifications by the hand of man, such as to prevent cross-pairing with endogenous TCR when present in a cell and/or to enhance heterologous pairing with each other. In specific embodiments, the α chain and/or β chain are not from a human but are from another mammal, such as a mouse, rat, or monkey, for example. In specific embodiments, both V_{L} and V_{H} of the anti-TdT peptide antibody are attached to the same TCR chain (a or b) (FIG. 7). In some embodiments, both TCR chains are attached to either V_{H} or V_{L} of the anti-TdT peptide antibody. In specific embodiments, representative sequences may be employed in the compositions of the disclosure, where the bold underlined sequences have been modified compared to wild-type:

TCR alpha:
(SEQ ID NO: 1)
PYIQNPEPAVYQLKDPRSQDSTLCLFTDFDSQINVPKTMESGTFIT
DKCVLDMKAMDSKSNGAIAWSNQTSFTCQDIFKETNATYPSSDVPC
DATLTEKSFETDMNLNFQNLLVIVLRILLLKVAGFNLLMTLRLWSS
TCR beta:
(SEQ ID NO: 2)
EDLRNVTPPKVSLFEPSKAEIANKQKATLVCLARGFFPDHVELSWW
VNGKEVHSGVCTDPQAYKESNYSYCLSSRLRVSATFWHNPRNHFRC
QVQFHGLSEEDKWPEGSPKPVTQNISAEAWGRADCGITSASYQQGV
LSATILYEILLGKATLYAVLVSTLVVMAMVKRKNS

Any protein, polynucleotide, and/or cells encompassed herein may be comprised in a composition. Cells in a composition may be of any kind, including immune cells, such as immune effector cells. The cells in a composition may be T cells, NK cells, NK T cells, B cells, macrophages, neutrophils, or a combination thereof. When the cell are T cells, the T cells may be ab T cells, gd T cells, T-helper cells, invariant natural killer T (iNKT) cells, cytotoxic T cells, T-regulatory cells natural-killer (NK) cells, or a combination thereof. In various embodiments, the cells may comprise (a) a polynucleotide comprising (1) sequence that encodes a recombinant TCR comprising an scFv specific for TdT or a TdT peptide; and (2) sequence that encodes a BiTE comprising an antibody specific for TdT or a TdT peptide; or (b) a first polynucleotide comprising sequence that encodes a recombinant TCR comprising an scFv specific for TdT or a TdT peptide; and a second polynucleotide comprising sequence that encodes a BiTE comprising an antibody specific for TdT or a TdT peptide.

### B. Types of Antibodies

The TdT-specific antibody may be of any type. Antibodies can be whole immunoglobulins of any isotype or classification, chimeric antibodies, or hybrid antibodies with specificity to two or more antigens. They may also be fragments (e.g., F(ab′)₂, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins.

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bi-specific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with V_{H} and V_{L} region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the V_{H} and V_{L} domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, VH and VL, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the VL domain are identified as L1, L2, and L3, with L1 occurring at the most distal end and L3 occurring closest to the CL domain. The CDRs may also be given the names CDR-L1, CDR-L2, and CDR-L3. The L3 (CDR-L3) is generally the region of highest variability among all antibody molecules produced by a given organism. The CDRs are regions of the polypeptide chain arranged linearly in the primary structure, and separated from each other by Framework Regions. The amino terminal (N-terminal) end of the VL chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the VH chain, which includes three CDRs identified as CDR-H1, CDR-H2 and CDR-H3. The majority of amino acid residues in the variable domains, or Fv fragments (VH and VL), are part of the framework regions (approximately 85%). The three dimensional, or tertiary, structure of an antibody molecule is such that the framework regions are more internal to the molecule and provide the majority of the structure, with the CDRs on the external surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include:

1) Computational predictions of the tertiary structure of the antibody/epitope binding interactions based on the chemical nature of the amino acid sequence of the antibody variable region and composition of the epitope.

2) Hydrogen-deuterium exchange and mass spectroscopy

3) Polypeptide fragmentation and peptide mapping approaches in which one generates multiple overlapping peptide fragments from the full length of the polypeptide and evaluates the binding affinity of these peptides for the epitope.

4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific VH and VL domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by VH and VL domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins are the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes chimeric antibody function and reduces immunogenicity. Specific amino acid residues from non-antigen recognizing regions of the non-human antibody are modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### C. Functional Antibody Fragments and Antigen-Binding Fragments

### 1. Antigen-Binding Fragments

Certain aspects relate to antibody fragments, such as antibody fragments that bind to a TdT peptide. The term functional antibody fragment includes antigen-binding fragments of an antibody that retain the ability to specifically bind to an antigen. These fragments are constituted of various arrangements of the variable region heavy chain (VH) and/or light chain (VL); and in some embodiments, include constant region heavy chain 1 (CH1) and light chain (CL). In some embodiments, they lack the Fc region constituted of heavy chain 2 (CH2) and 3 (CH3) domains. Embodiments of antigen binding fragments and the modifications thereof may include: (i) the Fab fragment type constituted with the VL, VH, CL, and CH1 domains; (ii) the Fd fragment type constituted with the VH and CH1 domains; (iii) the Fv fragment type constituted with the VH and VL domains; (iv) the single domain fragment type, dAb, (Ward, 1989; McCafferty et al., 1990; Holt et al., 2003) constituted with a single VH or VL domain; (v) isolated complementarity determining region (CDR) regions. Such terms are described, for example, in Harlow and Lane, Antibodies: A Laboratory Manual, Cold Spring Harbor Laboratory, NY (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015), each of which are incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a CH2 or CH3 region thereof) are included within the scope of this definition including, for example, scFv fused, directly or indirectly, to an Fc region are included herein.

The term Fab fragment (also “Fab”) means a monovalent antigen-binding fragment of an antibody containing the VL, VH, CL and CH1 domains. The term Fab′ fragment means a monovalent antigen-binding fragment of a monoclonal antibody that is larger than a Fab fragment. For example, a Fab′ fragment includes the VL, VH, CL and CH1 domains and all or part of the hinge region. The term F(ab′)₂ fragment means a bivalent antigen-binding fragment of a monoclonal antibody comprising two Fab′ fragments linked by a disulfide bridge at the hinge region. An F(ab′)₂ fragment includes, for example, all or part of the two VH and VL domains, and can further include all or part of the two CL and CH1 domains.

The term Fd fragment means a fragment of the heavy chain of a monoclonal antibody, which includes all or part of the VH, including the CDRs. An Fd fragment can further include CH1 region sequences.

The term Fv fragment means a monovalent antigen-binding fragment of a monoclonal antibody, including all or part of the VL and VH, and absent of the CL and CH1 domains. The VL and VH include, for example, the CDRs. Single-chain antibodies (sFv or scFv) are Fv molecules in which the VL and VH regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)₂ means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)₂ fragments are also known as “miniantibodies” or “minibodies.”

A single domain antibody is an antigen-binding fragment containing only a VH or the VL domain. In some instances, two or more VH regions are covalently joined with a peptide linker to create a bivalent domain antibody. The two VH regions of a bivalent domain antibody may target the same or different antigens.

### 2. Fragment Antigen Binding Region, Fab

Fab polypeptides of the disclosure include the Fab antigen binding fragment of an antibody. Unless specifically stated otherwise, the term “Fab” relates to a polypeptide excluding the Fc portion of the antibody. The Fab may be conjugated to a polypeptide comprising other components, such as further antigen binding domains, costimulatory domains, linkers, peptide spacers, transmembrane domains, endodomains, and accessory proteins. Fab polypeptides can be generated using conventional techniques known in the art and are well-described in the literature.

### 3. Fragment Crystallizable Region, Fe

An Fc region contains two heavy chain fragments comprising the CH2 and CH3 domains of an antibody. The two heavy chain fragments are held together by two or more disulfide bonds and by hydrophobic interactions of the CH3 domains. The term “Fc polypeptide” as used herein includes native and mutein forms of polypeptides derived from the Fc region of an antibody. Truncated forms of such polypeptides containing the hinge region that promotes dimerization are included.

D. Polypeptides with antibody CDRs & Scaffolding Domains that Display the CDRs

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### E. Antibody Binding

The term “selective binding agent” refers to a molecule that binds to an antigen. Non-limiting examples include antibodies, antigen-binding fragments, scFv, Fab, Fab′, F(ab′)₂, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

### 1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. Affinity can be expressed as, for example, 20-fold greater binding ability of the antibody to its antigen then to an unrelated amino acid sequence. As used herein, the term “avidity” refers to the resistance of a complex of two or more agents to dissociation after dilution. The terms “immunoreactive” and “preferentially binds” are used interchangeably herein with respect to antibodies and/or selective binding agent.

There are several experimental methods that can be used by one skilled in the art to evaluate the binding affinity of any given antibody or selective binding agent for its antigen. This is generally done by measuring the equilibrium dissociation constant (KD or Kd), using the equation KD=koff/kon=[A][B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the KD are mol/L (molarity, or M), or concentration. The Ka of an antibody is the opposite of the KD, and is determined by the equation Ka=1/KD. Examples of some experimental methods that can be used to determine the KD value are: enzyme-linked immunosorbent assays (ELISA), isothermal titration calorimetry (ITC), fluorescence anisotropy, surface plasmon resonance (SPR), and affinity capillary electrophoresis (ACE). The affinity constant (Ka) of an antibody is the opposite of the KD, and is determined by the equation Ka=1/KD.

Antibodies deemed useful in certain embodiments may have an affinity constant (Ka) of about, at least about, or at most about 10⁶, 10⁷, 10⁸, 10⁹, or 10¹⁰ M or any range derivable therein. Similarly, in some embodiments, antibodies may have a dissociation constant of about, at least about or at most about 10⁻⁶, 10⁻⁷, 10⁻⁸, 10⁻⁹, 10⁻¹⁰ M, or any range derivable therein. These values are reported for antibodies discussed herein and the same assay may be used to evaluate the binding properties of such antibodies. An antibody of the invention is said to “specifically bind” its target antigen when the dissociation constant (KD) is ≤10⁻⁸ M. The antibody specifically binds antigen with “high affinity” when the KD is ≤5×10⁻⁹ M, and with “very high affinity” when the KD is 5×10⁻¹⁰ M.

### 2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cells respond or recognize. Polypeptide epitopes can be formed from both contiguous amino acids and noncontiguous amino acids juxtaposed by tertiary folding of a polypeptide. An epitope typically includes at least 3, and typically 5-10 amino acids in a unique spatial conformation.

Epitope specificity of an antibody can be determined in a variety of ways. One approach, for example, involves testing a collection of overlapping peptides of 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

### 3. Modification of Antibody Antigen-Binding Domains

It is understood that the antibodies of the present disclosure may be modified, such that they are substantially identical to the antibody polypeptide sequences, or fragments thereof, and still bind the epitopes of the present invention. Polypeptide sequences are “substantially identical” when optimally aligned using such programs as Clustal Omega, IGBLAST, GAP or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present invention, providing that the variations in the amino acid sequence maintain at least 75%, more preferably at least 80%, at least 90%, at least 95%, at least 96%, at least 97%, at least 98% and most preferably at least 99% sequence identity. In particular, conservative amino acid replacements are contemplated.

Conservative replacements are those that take place within a family of amino acids that are related in their side chains. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). For example, it is reasonable to expect that an isolated replacement of a leucine moiety with an isoleucine or valine moiety, or a similar replacement of an amino acid with a structurally related amino acid in the same family, will not have a major effect on the binding or properties of the resulting molecule, especially if the replacement does not involve an amino acid within a framework site. Whether an amino acid change results in a functional peptide can readily be determined by assaying the specific activity of the polypeptide derivative. Standard ELISA, Surface Plasmon Resonance (SPR), or other antibody binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum., Science 338:1042-1046 (2012). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

Framework modifications can be made to antibodies to decrease immunogenicity, for example, by “backmutating” one or more framework residues to a corresponding germline sequence.

It is also contemplated that the antigen-binding domain may be multi-specific or multivalent by multimerizing the antigen-binding domain with VH and VL region pairs that bind either the same antigen (multi-valent) or a different antigen (multi-specific).

### F. Chemical Modification of Antibodies

In some aspects, also contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861). In certain embodiments, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other embodiments, one or more new N-linked glycosylation sites are created. Antibodies typically have an N-linked glycosylation site in the Fc region.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

In some aspects, the polypeptides can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive water-soluble polymer). Methods for pegylating proteins are known in the art and can be applied to the polypeptides of the invention to obtain PEGylated derivatives of antibodies. See, e.g., EP 0 154 316 and EP 0 401 384. In some aspects, the antibody is conjugated or otherwise linked to transthyretin (TTR) or a TTR variant. The TTR or TTR variant can be chemically modified with, for example, a chemical selected from the group consisting of dextran, poly(n-vinyl pyrrolidone), polyethylene glycols, propropylene glycol homopolymers, polypropylene oxide/ethylene oxide co-polymers, polyoxyethylated polyols, and polyvinyl alcohols. As used herein, the term “polyethylene glycol” is intended to encompass any of the forms of PEG that have been used to derivatize other proteins.

### 1. Conjugation

Derivatives of the antibodies and antigen binding fragments that are described herein are also provided. The derivatized antibody or fragment thereof may comprise any molecule or substance that imparts a desired property to the antibody or fragment. The derivatized antibody can comprise, for example, a detectable (or labeling) moiety (e.g., a radioactive, colorimetric, antigenic, or enzymatic molecule, or a detectable bead), a molecule that binds to another molecule (e.g., biotin or streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses).

Optionally, an antibody or an immunological portion of an antibody can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0 486 525. In some aspects, the polypeptides may be conjugated to a diagnostic agent and used diagnostically, for example, to monitor the development or progression of a disease and determine the efficacy of a given treatment regimen. In some aspects, the polypeptides may also be conjugated to a therapeutic agent to provide a therapy in combination with the therapeutic effect of the polypeptide. Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. The functional nucleic acid molecules may act as effectors, inhibitors, modulators, and stimulators of a specific activity possessed by a target molecule, or the functional nucleic acid molecules may possess a de novo activity independent of any other molecules.

In some aspects, disclosed are antibodies and antibody-like molecules that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

### 2. Conjugate Types

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRP), fluorescein, FITC, rhodamine, dansyl, umbelliferone, dimethyl acridinium ester (DMAE), Texas red, luminol, NADPH and α- or β-galactosidase. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference. Molecules containing azido groups may also be used to form covalent bonds to proteins through reactive nitrene intermediates that are generated by low intensity ultraviolet light (Potter & Haley, 1983).

In some aspects, contemplated are immunoconjugates comprising an antibody or antigen-binding fragment thereof conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing cell surface antigen. The antibody and agent may be associated through non-covalent interactions such as through electrostatic forces, or by covalent bonds. Various linkers, known in the art, can be employed in order to form the immunoconjugate. Additionally, the immunoconjugate can be provided in the form of a fusion protein. In one aspect, an antibody may be conjugated to various therapeutic substances in order to target the cell surface antigen. Examples of conjugated agents include, but are not limited to, metal chelate complexes, drugs, toxins and other effector molecules, such as cytokines, lymphokines, chemokines, immunomodulators, radiosensitizers, asparaginase, carboranes, and radioactive halogens.

In antibody drug conjugates (ADC), an antibody (Ab) is conjugated to one or more drug moieties (D) through a linker (L). The ADC may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Antibody drug conjugates may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate. In yet another aspect, the antibody may be conjugated to a “receptor” (such as streptavidin) for utilization in tumor or cancer cell pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

Examples of an antibody-drug conjugates known to a person skilled in the art are pro-drugs useful for the local delivery of cytotoxic or cytostatic agents, i.e. drugs to kill or inhibit tumor cells in the treatment of cancer (Syrigos and Epenetos, Anticancer Res. 19:605-614 (1999); Niculescu-Duvaz and Springer, Adv. Drg. Del. Rev. 26:151-172 (1997); U.S. Pat. No. 4,975,278). In contrast, systematic administration of these unconjugated drug agents may result in unacceptable levels of toxicity to normal cells as well as the target tumor cells (Baldwin et al., Lancet 1:603-5 (1986); Thorpe, (1985) “Antibody Carriers of Cytotoxic Agents in Cancer Therapy: A Review,” In: Monoclonal Antibodies '84: Biological and Clinical Applications, A. Pincera et al., (eds.) pp. 475-506). Both polyclonal antibodies and monoclonal antibodies have been reported as useful in these strategies (Rowland et al., Cancer Immunol. Immunother. 21:183-87 (1986)).

In certain aspects, ADC include covalent or aggregative conjugates of antibodies, or antigen-binding fragments thereof, with other proteins or polypeptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG® (Sigma-Aldrich, St. Louis, Mo.) peptide as described in Hopp et al., Bio/Technology 6:1204 (1988), and U.S. Pat. No. 5,011,912. Oligomers that contain one or more antibody polypeptides may be employed as antagonists. Oligomers may be in the form of covalently linked or non-covalently linked dimers, trimers, or higher oligomers. Oligomers comprising two or more antibody polypeptides are contemplated for use. Other oligomers include heterodimers, homotrimers, heterotrimers, homotetramers, heterotetramers, etc. In certain aspects, oligomers comprise multiple antibody polypeptides joined via covalent or non-covalent interactions between peptide moieties fused to the antibody polypeptides. Such peptides may be peptide linkers (spacers), or peptides that have the property of promoting oligomerization. Leucine zippers and certain polypeptides derived from antibodies are among the peptides that can promote oligomerization of antibody polypeptides attached thereto, as described in more detail below.

### 3. Conjugation Methodology

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bos(p-diazoniumbenzoyl)-ethylenediamine), diisocyanates (such as toluene 2,6-diisocyanate), and bis-active fluorine compounds (such as 1,5-difluoro-2,4-dinitrobenzene). In some aspects, derivatization of immunoglobulins by selectively introducing sulfhydryl groups in the Fc region of an immunoglobulin, using reaction conditions that do not alter the antibody combining site, are contemplated. Antibody conjugates produced according to this methodology are disclosed to exhibit improved longevity, specificity, and sensitivity (U.S. Pat. No. 5,196,066, incorporated herein by reference). Site-specific attachment of effector or reporter molecules, wherein the reporter or effector molecule is conjugated to a carbohydrate residue in the Fc region has also been disclosed in the literature (O'Shannessy et al., 1987).

### III. Antibody Production

### A. Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745 (see, e.g., Antibodies: A Laboratory Manual, Cold Spring Harbor Laboratory, 1988; incorporated herein by reference). These antibodies may be polyclonal or monoclonal antibody preparations, monospecific antisera, human antibodies, hybrid or chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various embodiments can also be synthesized in solution or on a solid support in accordance with conventional techniques. See, for example, Stewart and Young, (1984); Tarn et al, (1983); Merrifield, (1986); and Barany and Merrifield (1979), each incorporated herein by reference.

Briefly, a polyclonal antibody is prepared by immunizing an animal with an antigen or a portion thereof and collecting antisera from that immunized animal. The antigen may be altered compared to an antigen sequence found in nature. In some embodiments, a variant or altered antigenic peptide or polypeptide is employed to generate antibodies. Inocula are typically prepared by dispersing the antigenic composition in a physiologically tolerable diluent to form an aqueous composition. Antisera is subsequently collected by methods known in the arts, and the serum may be used as-is for various applications or else the desired antibody fraction may be purified by well-known methods, such as affinity chromatography (Harlow and Lane, Antibodies: A Laboratory Manual 1988).

Methods of making monoclonal antibodies are also well known in the art (Kohler and Milstein, 1975; Harlow and Lane, 1988, U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide or domain. Resulting antibody-producing B-cells from the immunized animal, or all dissociated splenocytes, are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive. Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after two to three weeks) for the desired reactivity. Fusion procedures for making hybridomas, immunization protocols, and techniques for isolation of immunized splenocytes for fusion are known in the art.

Other techniques for producing monoclonal antibodies include the viral or oncogenic transformation of B-lymphocytes, a molecular cloning approach may be used to generate a nucleic acid or polypeptide, the selected lymphocyte antibody method (SLAM) (see, e.g., Babcook et al., Proc. Natl. Acad. Sci. USA 93:7843-7848 (1996), the preparation of combinatorial immunoglobulin phagemid libraries from RNA isolated from the spleen of the immunized animal and selection of phagemids expressing appropriate antibodies, or producing a cell expressing an antibody from a genomic sequence of the cell comprising a modified immunoglobulin locus using Cre-mediated site-specific recombination (see, e.g., U.S. Pat. No. 6,091,001).

Monoclonal antibodies may be further purified using filtration, centrifugation, and various chromatographic methods such as HPLC or affinity chromatography. Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used in accordance with embodiments include, but are not limited to, IL-1, IL-2, IL-4, IL-7, IL-12, -interferon, GMCSP, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m2) (Johnson/Mead, NJ), cytokines such as 0-interferon, IL-2, or IL-12, or genes encoding proteins involved in immune helper functions, such as B-7.A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991).

### B. Fully Human Antibody Production

Methods are available for making fully human antibodies. Using fully human antibodies can minimize the immunogenic and allergic responses that may be caused by administering non-human monoclonal antibodies to humans as therapeutic agents. In one embodiment, human antibodies may be produced in a non-human transgenic animal, e.g., a transgenic mouse capable of producing multiple isotypes of human antibodies to protein (e.g., IgG, IgA, and/or IgE) by undergoing V-D-J recombination and isotype switching. Accordingly, this aspect applies to antibodies, antibody fragments, and pharmaceutical compositions thereof, but also non-human transgenic animals, B-cells, host cells, and hybridomas that produce monoclonal antibodies. Applications of human antibodies include, but are not limited to, detect a cell expressing an anticipated protein, either in vivo or in vitro, pharmaceutical preparations containing the antibodies of the present invention, and methods of treating disorders by administering the antibodies.

Fully human antibodies can be produced by immunizing transgenic animals (usually mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., Proc. Natl. Acad. Sci. USA 90:2551-2555 (1993); Jakobovits et al., Nature 362:255-258 (1993); Bruggermann et al., Year in Immunol. 7:33 (1993). In one example, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then crossbred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, International Patent Application Publication Nos. WO 96/33735 and WO 94/02602, which are hereby incorporated by reference in their entirety. Additional methods relating to transgenic mice for making human antibodies are described in U.S. Pat. Nos. 5,545,807; 6,713,610; 6,673,986; 6,162,963; 6,300,129; 6,255,458; 5,877,397; 5,874,299 and 5,545,806; in International Patent Application Publication Nos. WO 91/10741 and WO 90/04036; and in European Patent Nos. EP 546073B1 and EP 546073A1, all of which are hereby incorporated by reference in their entirety for all purposes.

The transgenic mice described above, referred to herein as “HuMAb” mice, contain a human immunoglobulin gene minilocus that encodes unrearranged human heavy (μ and γ) and κ light chain immunoglobulin sequences, together with targeted mutations that inactivate the endogenous μ and κ chain loci (Lonberg et al., Nature 368:856-859 (1994)). Accordingly, the mice exhibit reduced expression of mouse IgM or κ chains and in response to immunization, the introduced human heavy and light chain transgenes undergo class switching and somatic mutation to generate high affinity human IgG κ monoclonal antibodies (Lonberg et al., supra; Lonberg and Huszar, Intern. Ref. Immunol. 13:65-93 (1995); Harding and Lonberg, Ann. N.Y. Acad. Sci. 764:536-546 (1995)). The preparation of HuMAb mice is described in detail in Taylor et al., Nucl. Acids Res. 20:6287-6295 (1992); Chen et al., Int. Immunol. 5:647-656 (1993); Tuaillon et al., J. Immunol. 152:2912-2920 (1994); Lonberg et al., supra; Lonberg, Handbook of Exp. Pharmacol. 113:49-101 (1994); Taylor et al., Int. Immunol. 6:579-591 (1994); Lonberg and Huszar, Intern. Ref. Immunol. 13:65-93 (1995); Harding and Lonberg, Ann. N.Y. Acad. Sci. 764:536-546 (1995); Fishwild et al., Nat. Biotechnol. 14:845-851 (1996); the foregoing references are herein incorporated by reference in their entirety for all purposes. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807; as well as International Patent Application Publication Nos. WO 93/1227; WO 92/22646; and WO 92/03918, the disclosures of all of which are hereby incorporated by reference in their entirety for all purposes. Technologies utilized for producing human antibodies in these transgenic mice are disclosed also in WO 98/24893, and Mendez et al., Nat. Genetics 15:146-156 (1997), which are herein incorporated by reference. For example, the HCo7 and HCo12 transgenic mice strains can be used to generate human antibodies.

Using hybridoma technology, antigen-specific humanized monoclonal antibodies with the desired specificity can be produced and selected from the transgenic mice such as those described above. Such antibodies may be cloned and expressed using a suitable vector and host cell, or the antibodies can be harvested from cultured hybridoma cells. Fully human antibodies can also be derived from phage-display libraries (as disclosed in Hoogenboom et al., J. Mol. Biol. 227:381 (1991); and Marks et al., J. Mol. Biol. 222:581 (1991)). One such technique is described in International Patent Application Publication No. WO 99/10494 (herein incorporated by reference), which describes the isolation of high affinity and functional agonistic antibodies for MPL- and msk-receptors using such an approach.

### C. Antibody Fragments Production

Antibody fragments that retain the ability to recognize the antigen of interest will also find use herein. A number of antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv. See, e.g., Inbar et al., Proc. Nat. Acad. Sci. USA 69:2659-2662 (1972); Hochman et al., Biochem. 15:2706-2710 (1976); and Ehrlich et al., Biochem. 19:4091-4096 (1980).

Single-chain variable fragments (scFvs) may be prepared by fusing DNA encoding a peptide linker between DNAs encoding the two variable domain polypeptides (VL and VH). scFvs can form antigen-binding monomers, or they can form multimers (e.g., dimers, trimers, or tetramers), depending on the length of a flexible linker between the two variable domains (Kortt et al., Prot. Eng. 10:423 (1997); Kort et al., Biomol. Eng. 18:95-108 (2001)). By combining different VL- and VH-comprising polypeptides, one can form multimeric scFvs that bind to different epitopes (Kriangkum et al., Biomol. Eng. 18:31-40 (2001)). Antigen-binding fragments are typically produced by recombinant DNA methods known to those skilled in the art. Although the two domains of the Fv fragment, VL and VH, are coded for by separate genes, they can be joined using recombinant methods by a synthetic linker that enables them to be made as a single chain polypeptide (known as single chain Fv (sFv or scFv); see e.g., Bird et al., Science 242:423-426 (1988); and Huston et al., Proc. Natl. Acad. Sci. USA 85:5879-5883 (1988). Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues, and may include glutamic acid and lysine residues inserted to enhance solubility. Antigen-binding fragments are screened for utility in the same manner as intact antibodies. Such fragments include those obtained by amino-terminal and/or carboxy-terminal deletions, where the remaining amino acid sequence is substantially identical to the corresponding positions in the naturally occurring sequence deduced, for example, from a full-length cDNA sequence.

Antibodies may also be generated using peptide analogs of the epitopic determinants disclosed herein, which may consist of non-peptide compounds having properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al., J. Med. Chem. 30:1229 (1987). Liu et al. (2003) also describe “antibody like binding peptidomimetics” (ABiPs), which are peptides that act as pared-down antibodies and have certain advantages of longer serum half-life as well as less cumbersome synthesis methods. These analogs can be peptides, non-peptides or combinations of peptide and non-peptide regions. Fauchere, Adv. Drug Res. 15:29 (1986); Veber and Freidiner, TINS p. 392 (1985); and Evans et al., J. Med. Chem. 30:1229 (1987), which are incorporated herein by reference in their entirety for any purpose. Peptide mimetics that are structurally similar to therapeutically useful peptides may be used to produce a similar therapeutic or prophylactic effect. Such compounds are often developed with the aid of computerized molecular modeling. Generally, peptidomimetics of the invention are proteins that are structurally similar to an antibody displaying a desired biological activity, such as the ability to bind a protein, but have one or more peptide linkages optionally replaced by a linkage selected from: —CH2NH—, —CH₂S—, —CH2-CH2-, —CH═CH— (cis and trans), COCH2, —CH(OH)CH2-, and —CH2SO— by methods well known in the art. Systematic substitution of one or more amino acids of a consensus sequence with a D-amino acid of the same type (e.g., D-lysine in place of L-lysine) may be used in certain embodiments of the invention to generate more stable proteins. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference), for example, by adding internal cysteine residues capable of forming intramolecular disulfide bridges which cyclize the peptide.

Once generated, a phage display library can be used to improve the immunological binding affinity of the Fab molecules using known techniques. See, e.g., Figini et al., J. Mol. Biol. 239:68 (1994). The coding sequences for the heavy and light chain portions of the Fab molecules selected from the phage display library can be isolated or synthesized and cloned into any suitable vector or replicon for expression. Any suitable expression system can be used.

### IV. Obtaining Encoded Antibodies

In some aspects, there are nucleic acid molecule encoding TdT peptide antibody polypeptides (e.g., heavy or light chain, variable domain only, or full-length). These may be generated by methods known in the art, e.g., isolated from B cells of mice that have been immunized and isolated, phage display, expressed in any suitable recombinant expression system and allowed to assemble to form antibody molecules.

### A. Expression

The nucleic acid molecules may be used to express large quantities of recombinant antibodies or to produce chimeric antibodies, single chain antibodies, immunoadhesins, diabodies, mutated antibodies, and other antibody derivatives. If the nucleic acid molecules are derived from a non-human, non-transgenic animal, the nucleic acid molecules may be used for antibody humanization.

### 1. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

To express the antibodies, or antigen-binding fragments thereof, DNAs encoding partial or full-length light and heavy chains are inserted into expression vectors such that the gene area is operatively linked to transcriptional and translational control sequences. In some aspects, a vector that encodes a functionally complete human CH or CL immunoglobulin sequence with appropriate restriction sites engineered so that any VH or VL sequence can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### 2. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### 3. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### 4. Host Cells

In another aspect, contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### B. Isolation

The nucleic acid molecule encoding either or both of the entire heavy and light chains of an antibody or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding the full-length heavy and/or light chains may then be expressed in a cell into which they have been introduced and the antibody isolated.

### II. Antibodies, Antigen Binding Fragments, and Polypeptides

The present disclosure encompasses antibody proteins or polypeptides of any kind and chimeric protein or polypeptide molecules of any kind, including chimeric protein or polypeptide molecules that encompass a functional part or all of the antibody proteins or polypeptides. As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity. The term polypeptide also includes and antibody fragment described herein as well as antibody domains, such as HCDR1, HCDR2, HCDR3, LCDR1, LCDR2, LCDR3, HFRW1, HFRW2, HFRW3, HFRW4, LFRW1, LFRW2, LFRW3, LFRW4, V_{H}, V_{L}, CH, or CL.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of an antibody, antigen binding fragment, protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, or 450 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The antibody, antigen binding fragment, polypeptides, proteins, or polynucleotides encoding such polypeptides or proteins of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, or more contiguous amino acids or nucleic acids, or any range derivable therein, of SEQ ID NO:4-27.

In some embodiments, the antibody, antigen binding fragment, protein, or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, (or any derivable range therein) of SEQ ID NOS:4-9 or -22-27.

In some embodiments, the antibody, antigen binding fragment, or polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425 (or any derivable range therein) contiguous amino acids or nucleic acids of SEQ ID NOs:4-9 or -22-27.

In some embodiments, the antibody, antigen binding fragment, protein, or polypeptide may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425 or more (or any derivable range therein) contiguous amino acids or nucleic acids of SEQ ID NOS: 4-9 or -22-27 that are at least, at most, or exactly 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOS: 4-9 or -22-27.

In some aspects there is a nucleic acid molecule, antibody, antigen binding fragment, protein, or polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOS:4-27 and comprising at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110,111, 112, 113, 114, 115, 116, 117, 118, 119, 120,121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOS:4-27.

In some embodiments, the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110,111, 112, 113, 114, 115, 116, 117, 118, 119, 120,121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 of the heavy chain, light chain, VH, VL, HCDR1, HCDR2, HCDR3, LCDR1, LCDR2, LCDR3, HFRW1, HFRW2, HFRW3, HFRW4, LFRW1, LFRW2, LFRW3, or LFRW4 encompassed herein is substituted with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

The nucleotide as well as the protein, polypeptide, and peptide sequences for various genes have been previously disclosed, and may be found in the recognized computerized databases. Two commonly used databases are the National Center for Biotechnology Information's Genbank and GenPept databases (on the World Wide Web at ncbi.nlm.nih.gov/) and The Universal Protein Resource (UniProt; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### V. Sequences

Polypeptide, antibody, and antigen binding fragment embodiments are encompassed herein.

The following is a discussion of changing the amino acid subunits of a protein to create an equivalent, or even improved, second-generation variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least 50%, 60%, 70%, 80%, or 90%, including all values and ranges there between, identical to any sequence provided or referenced herein. A variant can include 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the invention, those that are within ±1 are included, and in other aspects of the invention, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within ±2 are included, in other embodiments, those which are within ±1 are included, and in still other embodiments, those within ±0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the invention, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### III. Nucleic Acids

In certain embodiments, nucleic acid sequences can exist in a variety of instances such as: isolated segments and recombinant vectors of incorporated sequences or recombinant polynucleotides encoding peptides and polypeptides of the disclosure, or a fragment, derivative, mutein, or variant thereof, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

Changes can be introduced by mutation into a nucleic acid, thereby leading to changes in the amino acid sequence of a polypeptide (e.g., an antigenic peptide or polypeptide) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### VI. Polypeptide Expression

In some aspects, there are nucleic acid molecule encoding polypeptides, antibodies, or antigen binding fragments of the disclosure. The nucleic acid molecules may be used to express large quantities of polypeptides, including antibodies and/or chimeric polypeptides that use them. If the nucleic acid molecules are derived from a non-human, non-transgenic animal, the nucleic acid molecules may be used for humanization of the antibody or TCR genes.

### A. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody heavy and/or light chain, antibody fragments, and probes thereof. In addition to control sequences that govern transcription and translation, vectors and expression vectors may contain nucleic acid sequences that serve other functions as well.

To express the polypeptides or peptides of the disclosure, DNAs encoding the polypeptides or peptides are inserted into expression vectors such that the gene area is operatively linked to transcriptional and translational control sequences. In some aspects, a vector that encodes a functionally complete human CH or CL immunoglobulin sequence with appropriate restriction sites engineered so that any V_{H} or V_{L} sequence can be easily inserted and expressed. In some aspects, a vector that encodes a functionally complete human TCR alpha or TCR beta sequence with appropriate restriction sites engineered so that any variable sequence or CDR1, CDR2, and/or CDR3 can be easily inserted and expressed. Typically, expression vectors used in any of the host cells contain sequences for plasmid or virus maintenance and for cloning and expression of exogenous nucleotide sequences. Such sequences, collectively referred to as “flanking sequences” typically include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### B. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### C. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### VII. Methods of Treatment

In various embodiments, diseased or other cells expressing TdT peptide on their surface are targeted for the purpose of improving a medical condition in an individual that has the medical condition or for the purpose of reducing the risk or delaying the severity and/or onset of the medical condition in an individual. In specific cases, cancer cells expressing endogenous TdT are targeted for the purpose of killing the cancer cells.

TdT peptide-targeting antibody constructs, nucleic acid sequences, vectors, immune cells expressing same as contemplated herein, and/or pharmaceutical compositions comprising the same, are used for the prevention, treatment or amelioration of a cancerous disease, such as a tumorous disease. In particular embodiments, the pharmaceutical composition of the present disclosure may be particularly useful in preventing, ameliorating and/or treating cancer, including cancers that express TdT peptide and that may or may not be solid tumors, for example.

The administration of the composition(s) of the disclosure is useful for all stages (I, II, III, or IV) and types of cancer, including for minimal residual disease, early cancer, advanced cancer, and/or metastatic cancer and/or refractory cancer, for example.

The disclosure further encompasses co-administration protocols with other compounds, e.g. surgery, radiation, chemotherapy, other immunotherapy, drug therapy, or a hormone therapy, combination thereof. The clinical regimen for co-administration of the inventive compound(s) may encompass co-administration at the same time, before or after the administration of the other component. Particular combination therapies include chemotherapy, radiation, surgery, hormone therapy, or other types of immunotherapy.

### VIII. Methods of Evaluation, Detection, and Diagnosis

Embodiments of the disclosure encompass methods for evaluating any sample from a subject, the method comprising contacting a biological sample from the subject, or extract thereof, with at least one antibody, antigen binding fragment, or polypeptide encompassed herein. In various embodiments, methods of detecting cancer are provided that comprise contacting a biological sample from the subject, or extract thereof, with at least one antibody, antigen binding fragment, or polypeptide encompassed herein. In particular embodiments, there is a method for diagnosing cancer in a subject, the method comprising contacting a biological sample from the subject, or extract thereof, with at least one antibody, antigen binding fragment, or polypeptideln any evaluation method, the sample may be of any kind, but in specific embodiments it comprises a blood sample, urine sample, fecal sample, nasopharyngeal sample, cerebrospinal fluid sample, cheek scraping sample, nipple aspirate sample, biopsy sample, or a combination thereof.

In various embodiments for evaluation, detection, or diagnosis, the at least one antibody, antigen binding fragment, or polypeptide may be operatively linked to a detectable label. Any of the methods may further comprise incubating the antibody, antigen binding fragment, or polypeptide under conditions that allow for the binding of the antibody, antigen binding fragment, or polypeptide to antigens in the biological sample or extract thereof. The method may further comprise detecting the binding of an antigen to the antibody, antigen binding fragment, or polypeptide. The method may further comprise contacting the biological sample with at least one capture antibody, antigen, or polypeptide. The capture antibody may be linked to a solid support.

In specific embodiments, the at least one antibody, antigen binding fragment, or polypeptide is operatively linked to a detectable label. The method may further comprise incubating the antibody, antigen binding fragment, or polypeptide under conditions that allow for the binding of the antibody, antigen binding fragment, or polypeptide to antigens in the biological sample or extract thereof. In various embodiments, the method further comprises detecting the binding of an antigen to the antibody, antigen binding fragment, or polypeptide. The method may further comprise contacting the biological sample with at least one capture antibody, antigen, or polypeptide.

### IX. Pharmaceutical Compositions

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenterally, orthotopically, intradermally, subcutaneously, orally, transdermally, intramuscularly, intraperitoneally, intraperitoneally, intraorbitally, by implantation, by inhalation, intraventricularly, intranasally, intraarterially, intratracheally, intrapleurally, intratumorally, endoscopically, intralesionally, intracranially, percutaneously, regionally, systemically, by perfusion, in a tumor microenvironment, or by intravenous injection. In some embodiments, compositions of the present disclosure (e.g., compositions comprising TdT peptide-binding polypeptides) are administered to a subject intravenously.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most or at least 3, 4, 5, 6, 7, 8, 9, 10 or more. The administrations may range from 2-day to 12-week intervals, more usually from one to two week intervals.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

Pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (e.g., polypeptides of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of the antibody that is administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about M to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### X. Detectable Labels

In some aspects of this disclosure, it will be useful to detectably or therapeutically label an antibody of any kind, including at least a Fab polypeptide or protein G Fab-binding domain. Methods for conjugating polypeptides to these agents are known in the art. For the purpose of illustration only, polypeptides can be labeled with a detectable moiety such as a radioactive atom, a chromophore, a fluorophore, or the like. Such labeled polypeptides can be used for diagnostic techniques, either in vivo, or in an isolated test sample or in methods described herein.

As used herein, the term “label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition that is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6.sup.th ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6.sup.th ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/polypeptides, e.g., rhodamine/anti-rhodamine, biotin/avidin and biotin/strepavidin.

The coupling of polypeptides to low molecular weight haptens can increase the sensitivity of the antibody in an assay. The haptens can then be specifically detected by means of a second reaction. For example, it is common to use haptens such as biotin, which reacts avidin, or dinitrophenol, pyridoxal, and fluorescein, which can react with specific anti-hapten polypeptides. See, Harlow and Lane (1988) supra.

### XI. Sample Preparation

In certain aspects, methods involve obtaining or evaluating a sample from a subject using antibodies and antibody-related agents encompassed herein. The sample may include a sample obtained from any source including but not limited to blood, sweat, hair follicle, buccal tissue, tears, menses, feces, nipple aspirate, cerebrospinal fluid, sputum, cheek scrapings, and/or saliva. In certain aspects of the current methods, any medical professional such as a doctor, nurse or medical technician may obtain a biological sample for testing. Yet further, the biological sample can be obtained without the assistance of a medical professional. The sample may be obtained by the individual in need of sample analysis.

A sample may include but is not limited to, tissue, cells, or biological material from cells or derived from cells of a subject. The biological sample may be a heterogeneous or homogeneous population of cells or tissues. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a blood sample, urine sample, fecal sample, nasopharyngeal sample, cerebrospinal fluid sample, cheek scraping sample, nipple aspirate sample, biopsy sample, or a combination thereof.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a esophageal or a suspected esophageal tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### IV. Host Cells

As used herein, the terms “cell,” “cell line,” and “cell culture” may be used interchangeably. All of these terms also include both freshly isolated cells and ex vivo cultured, activated or expanded cells. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

In certain embodiments transfection can be carried out on any prokaryotic or eukaryotic cell. In some aspects electroporation involves transfection of a human cell. In other aspects electroporation involves transfection of an animal cell. In certain aspects transfection involves transfection of a cell line or a hybrid cell type. In some aspects the cell or cells being transfected are cancer cells, tumor cells or immortalized cells. In some instances tumor, cancer, immortalized cells or cell lines are induced and in other instances tumor, cancer, immortalized cells or cell lines enter their respective state or condition naturally. In certain aspects the cells or cell lines can be A549, B-cells, B16, BHK-21, C2C12, C6, CaCo-2, CAP/, CAP-T, CHO, CHO2, CHO-DG44, CHO-K1, COS-1, Cos-7, CV-1, Dendritic cells, DLD-1, Embryonic Stem (ES) Cell or derivative, H1299, HEK, 293, 293T, 293FT, Hep G2, Hematopoietic Stem Cells, HOS, Huh-7, Induced Pluripotent Stem (iPS) Cell or derivative, Jurkat, K562, L5278Y, LNCaP, MCF7, MDA-MB-231, MDCK, Mesenchymal Cells, Min-6, Monocytic cell, Neuro2a, NIH 3T3, NIH3T3L1, K562, NK-cells, NS0, Panc-1, PC12, PC-3, Peripheral blood cells, Plasma cells, Primary Fibroblasts, RBL, Renca, RLE, SF21, SF9, SH-SY5Y, SK-MES-1, SK—N—SH, SL3, SW403, Stimulus-triggered Acquisition of Pluripotency (STAP) cell or derivate SW403, T-cells, THP-1, Tumor cells, U20S, U937, peripheral blood lymphocytes, expanded T cells, hematopoietic stem cells, or Vero cells.

### V. Kits

Certain aspects of the present invention also concern kits containing compositions of the disclosure or compositions to implement methods of the disclosure. In some embodiments, kits can be used to detect the presence of a TdT peptide in a sample. In some embodiments, kits can be used to detect the presence of a TdT peptide in a sample by means of an antibody that binds TdT peptide on the surface of cancer cells. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, a kit contains one or more polypeptides capable of binding to a TdT peptide disclosed herein. For example, a kit may comprise at least 1, 2, 3, 4, 5, 6, 7, 8, 9, 10 or more Fabs disclosed herein for detecting a TdT peptide. In some embodiments, a kit comprises a detection pair. In some embodiments, a kit comprises an enzyme. In some embodiments, a kit comprises a substrate for an enzyme.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits may comprise any apparatus to obtain a sample from an individual.

Kits for using probes, synthetic nucleic acids, nonsynthetic nucleic acids, and/or inhibitors of the disclosure for prognostic or diagnostic applications are included as part of the disclosure. In certain aspects, negative and/or positive control nucleic acids, probes, and inhibitors are included in some kit embodiments.

Kits may further comprise instructions for use. For example, in some embodiments, a kit comprises instructions for detecting cancer in a sample.

It is contemplated that any method or composition described herein can be implemented with respect to any other method or composition described herein and that different embodiments may be combined. The claims originally filed are contemplated to cover claims that are multiply dependent on any filed claim or combination of filed claims.

### VI. Examples

The following examples are included to demonstrate certain embodiments of the disclosure. It should be appreciated by those of skill in the art that the techniques disclosed in the examples that follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the disclosure.

### Example 1

TdT-Specific Cytotoxic Receptors and Methods of their Use

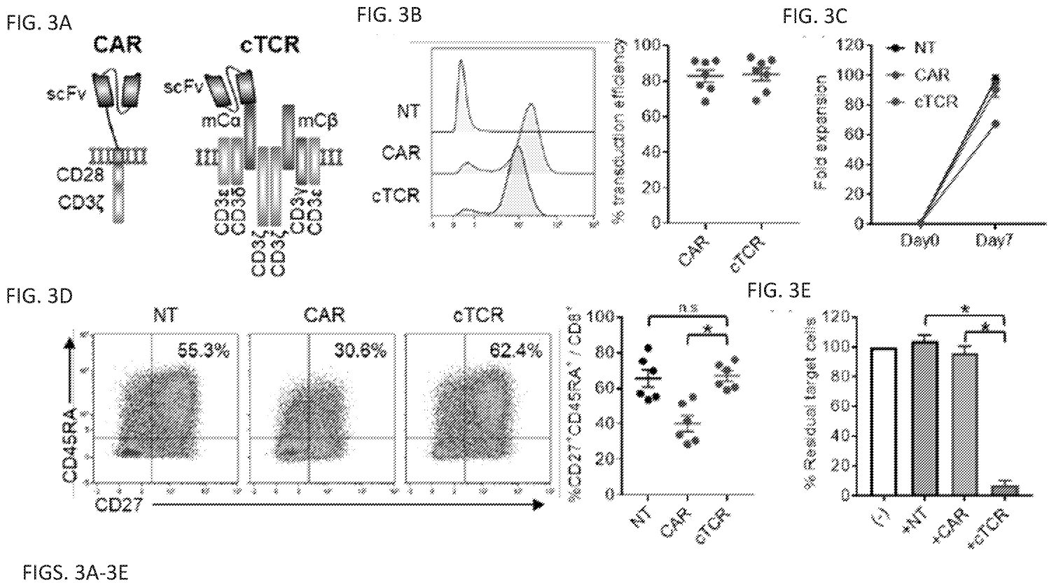

Terminal deoxynucleotidyl transferase (TdT) is a DNA polymerase expressed in T- and B-cell progenitors where it catalyzes transfer of nucleotides during DNA rearrangement (Kunkel et al., 1986; Desiderio et al., 1984). TdT is expressed in up to 95% of T- and B-lineage ALL and is widely used as a diagnostic marker for these malignancies (Drexler et al., 1986). In addition, TdT expression is frequently detected in minimally differentiated AML(Patel et al., 2012; Drexler et al., 1993). Off-target activity of TdT in immature progenitors has been linked with leukemogenes (Borrow et al., 2019a; Borrow et al., 2019b). Absence of TdT expression in primitive hematopoietic progenitors or mature peripheral lymphocytes makes it an ideal therapeutic target for leukemia. However, TdT-specific TCRs are effectively deleted in thymus, and intranuclear localization of TdT precludes its targeting with conventional CARs. Therefore, it was considered that TdT-expressing tumor cells can be targeted using a chimeric receptor that recognizes a TdT-derived peptide in the context of MHC I. A meta-analysis was performed of surface peptides presented by TdT+HLAA2+ leukemic cells and a TdT-derived peptide was identified that presented in the context of HLA-A02, a dominant MHC class I allele in the Western world (Klatt et al., 2020). Using recombinant HLA-A02:01/TdT peptide complexes, the inventors screened two separate phage display libraries comprised of >2×10¹² humanized scFv and humanized camelid VHH binders. Four clones that recognize TdT/HLA-A02:01 but not a control peptide/HLA-A02:01 complex have been identified. Because each individual peptide represents a very small fraction of total HLA-A molecules, surface density of TdT/HLA-A02 complexes is expected to be relatively low, possibly below the threshold of optimal CAR-mediated detection. To address this possibility, for each binder both second-generation TdT-specific CAR and TdT-specific chimeric TCR (cTCR) were generated to take advantage of the modular CAR structure and a highly sensitive TCR signaling, which is capable of detecting only a few cognate peptide/HLA molecules on the cell surface (Purbhoo et al., 2004; Sykuley et al., 1996) (FIG. 3A). In the cTCR construct, the TdT binders were fused with murine constant TCR α and/or β chains to enable specific dimerization and efficient integration with the CD3 signaling complex in human T-cells (FIG. 3A). Following gammaretroviral transduction, most TdT-specific receptors were expressed on the cell surface of primary human T-cells, with most binder variants demonstrating high level of expression (FIG. 3B) and expansion of transgenic T-cells (FIG. 3C). Expression of the TdT-specific CAR in T-cells resulted in a moderate reduction of minimally differentiated CD27+ CD45RA+ T-cells, likely due to tonic CAR signaling, whereas expression of TdT.cTCR did not affect T-cell differentiation status (FIG. 3D). Cytotoxicity of T-cells expressing TdT-specific constructs was evaluated in a 72-hour coculture with a TdT+HLA-A02+ leukemia cell line BV173. One TdT− specific cTCR construct produced robust cytotoxicity against leukemic cells whereas other clones exhibited reduced activity compared thereto (FIG. 3E). Notably, T-cells expressing a CAR with an identical TdT binder demonstrated only minimal activity suggesting CAR sensitivity was insufficient to elicit optimal T-cell activation.

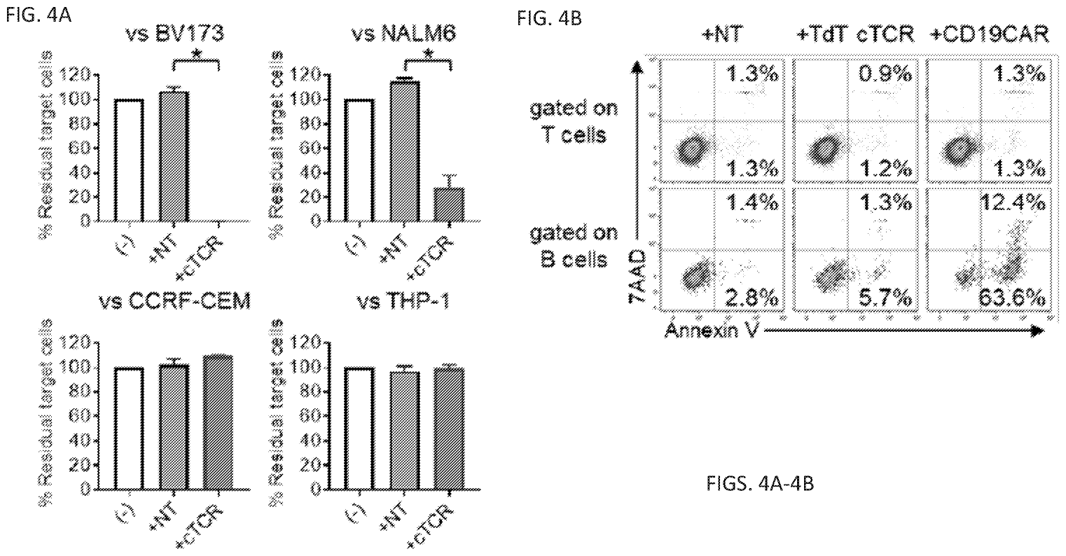

To assess specificity of cTCR T cells, 72-hour coculture assays were performed with malignant and normal lymphocytes and residual target cell number was quantified by flow cytometry with counting beads. TdT.cTCR T-cells produced high cytotoxicity against TdT+/HLA-A2+BV173 and NALM6 leukemia but demonstrated no activity against negative control CCRF-CEM (TdT+/HLA-A2−) and THP-1 (TdT-/HLA-A2+) cells (FIG. 4A) as well as against TdT-/HLA-A2+ cell lines GDM-1, Jeko-1, and Loucy. Next, TdT.cTCR T-cells were generated from HLA-A02+ donors and performed 24-hour coculture assays with autologous T- and B-cells freshly isolated from peripheral blood. No cytotoxicity against normal T- and B-lymphocytes was observed upon coculture with TdT.cTCR T-cells whereas control CD19 CAR T-cells expectedly killed autologous CD19+B-cells (FIG. 4B). Expression of TdT.cTCR on HLA-A02+ T-cells also did not produce fratricide and did not affect their ex vivo expansion. These data indicate that TdT.cTCR specifically recognizes TdT peptide in the context of HLA-A02 and does not elicit off-target activity against normal TdT-negative lymphocytes.

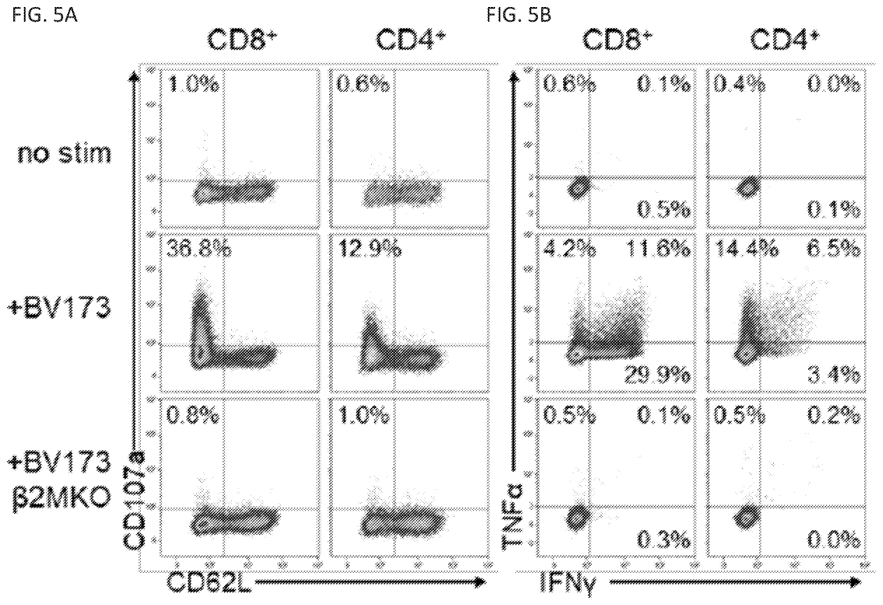

Because the TdT-specific binders identified in the phage display screening recognized TdT/HLA-A02 complexes without the help from the CD8 coreceptor, it was considered that TdT.cTCR would effectively redirect both CD4+ and CD8+ T-cells against leukemia. Indeed, there was robust degranulation (measured by CD107a staining) and production of IFNγ and TNFα by both CD4+ and CD8+ T-cells upon coculture with BV173 target cells (FIG. 5). This activity was abrogated by removing surface MHC class I expression using b2M CRISPR knockout indicating the activity of both CD4+ and CD8+TdT.cTCR T-cells was MHC class I-specific (FIG. 5). Therefore, unlike conventional tumor antigen-specific TCRs, TdT.cTCR can engage both CD4+ and CD8+ T-cell arms of immune response against leukemia.

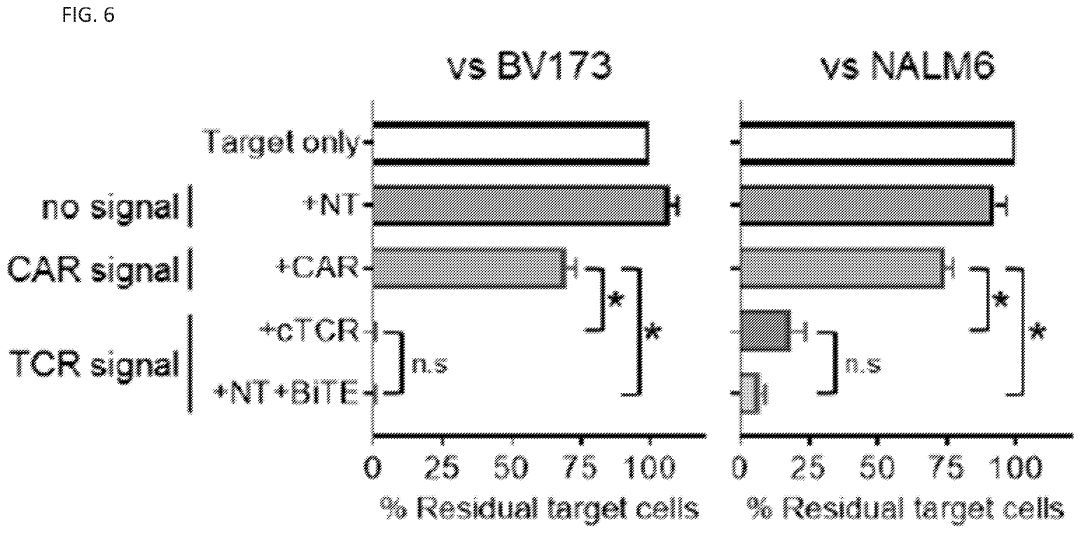

The results above indicate that TdT targeting is most effective by activating conventional TCR signaling. In addition to cTCR-transgenic Tcells, the TCR pathway can be engaged using a soluble bispecific T-cell engager (BiTE) where a tumor antigen-specific binder is fused with an anti-CD3 moiety thus inducing TCR crosslinking and T-cell activation/degranulation against the target cell. To test whether non-modified T-cells can be redirected against TdT+ leukemia with a BiTE, a TdT-specific engager was designed and expressed in producer cells. Standard 72-hour coculture assays were performed of unmodified non-transduced T-cells and TdT.CAR and TdT.cTCR T-cells against TdT+/HLA-A02+ leukemic cell lines BV173 and NALM-6. While control non-transduced T-cells had no measurable activity against leukemia in the absence of the BiTE, addition of soluble BiTE resulted in robust elimination of both leukemia targets by unmodified T-cells, on par with TdT.cTCR T-cells (FIG. 6). Collectively, these results demonstrate strong and specific anti-leukemic activity of TdT-specific cTCR and BiTE constructs compared to the CAR-based modality and support feasibility and efficacy of targeting TdT+ leukemia using these reagents.

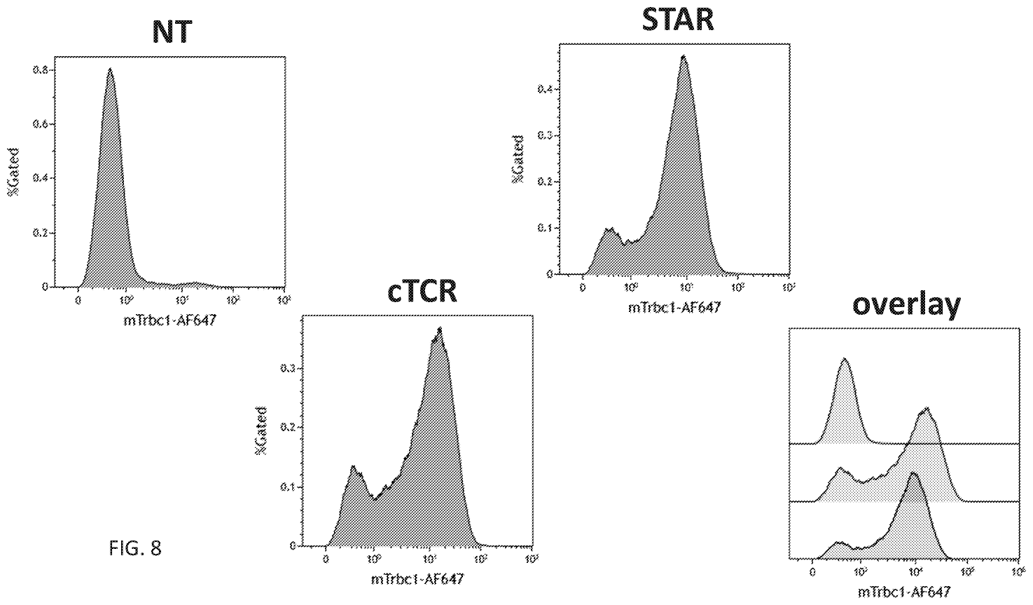

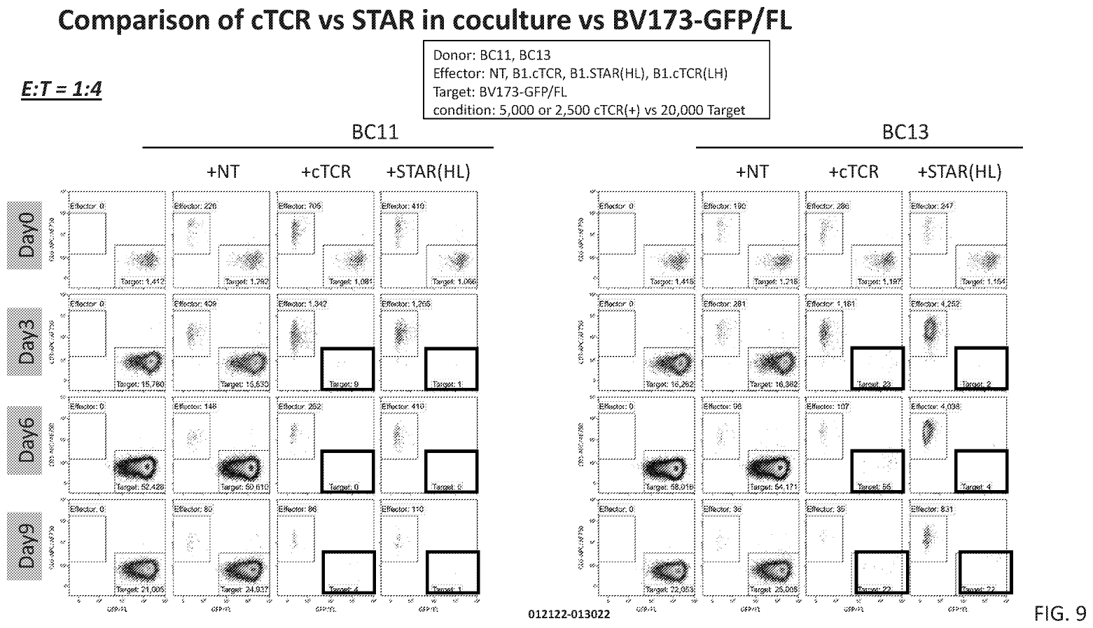

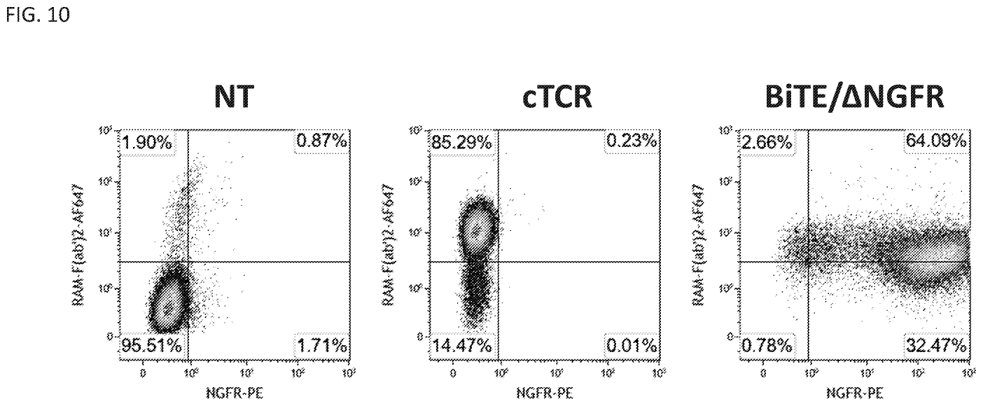

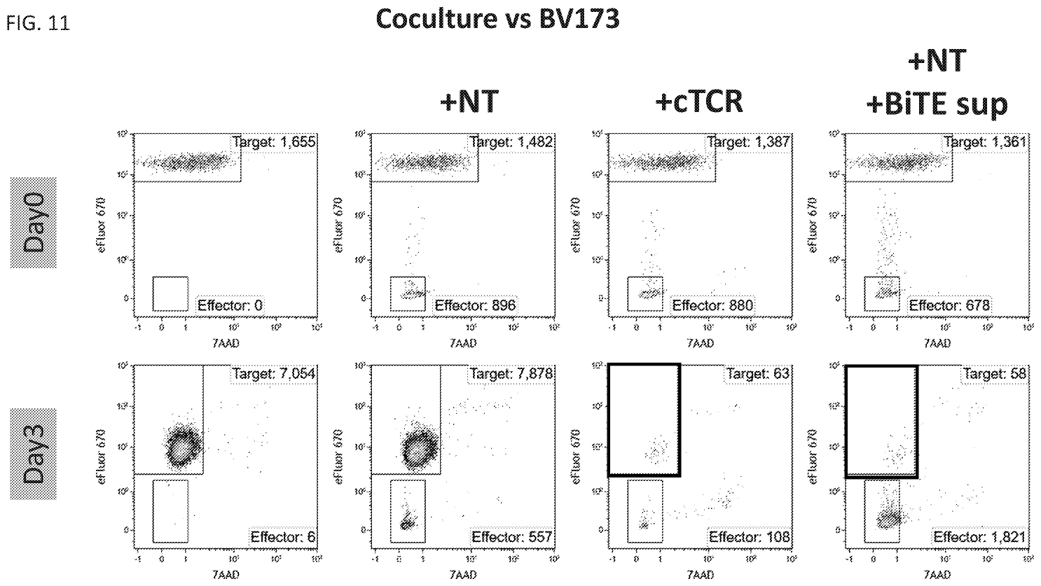

FIG. 7 illustrates one embodiment of a chimeric TCR in which V_{H} and V_{L} domains of the antibody are present on a single TCR α chain, in contrast to STAR receptors comprising the variable heavy and variable light regions on separate chains of a chimeric TCR (Liu et al., Sci Transl Med. 2021; 13(586):eabb5191). FIG. 8 shows cTCR and STAR expression on T cells after retroviral transduction. FIG. 9 demonstrates cytotoxicity of cTCR and STAR-expressing T cells against BV173 cell line (TdT+, HLA-A2+). In particular, the transduced cells were compared in co-culture vs. a representative cancer cell line of mixed-lineage leukemia (MLL) cells. In another embodiment, cTCR and BiTE molecules were transduced retrovirally into T cells (FIG. 10), and FIG. 11 provides evidence of cytotoxicity of the cTCR T cells and BiTE T cells against a representative cancer cell line of MLL cells (TdT+, HLA-A2+).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Borrow, J., Dyer, S. A., Akiki, S. & Griffiths, M. J. Terminal deoxynucleotidyl transferase promotes acute myeloid leukemia by priming FLT3-ITD replication slippage. Blood 134, 2281-2290 (2019a).
Borrow, J., Dyer, S. A., Akiki, S. & Griffiths, M. J. Molecular roulette: nucleophosmin mutations in AML are orchestrated through N-nucleotide addition by TdT. Blood 134, 2291-2303 (2019b).
Desiderio, S. V. et al. Insertion of N regions into heavy-chain genes is correlated with expression of terminal deoxytransferase in B cells. Nature 311, 752-755 (1984).
Drexler, H. G., Menon, M. & Minowada, J. Incidence of TdT positivity in cases of leukemia and lymphoma. Acta Haematol 75, 12-17 (1986).
Drexler, H. G., Sperling, C. & Ludwig, W. D. Terminal deoxynucleotidyl transferase (TdT) expression in acute myeloid leukemia. Leukemia 7, 1142-1150 (1993).
Gudipati, V. et al. Inefficient CAR-proximal signaling blunts antigen sensitivity. Nature Immunology 21, 848-856 (2020).
Klatt, M. G. et al. Solving an MHC allele-specific bias in the reported immunopeptidome. JCI Insight 5, (2020).
Kunkel, T. A., Gopinathan, K. P., Dube, D. K., Snow, E. T. & Loeb, L. A. Rearrangements of DNA mediated by terminal transferase. Proc Natl Acad Sci USA 83, 1867-1871 (1986).
Patel, J. L. et al. The immunophenotype of T-lymphoblastic lymphoma in children and adolescents: a Children's Oncology Group report. Br J Haematol 159, 454-461 (2012).
Purbhoo, M. A., Irvine, D. J., Huppa, J. B. & Davis, M. M. T cell killing does not require the formation of a stable mature immunological synapse. Nat Immunol 5, 524-530 (2004).
Sykulev, Y., Joo, M., Vturina, I. & Eisen, H. N. Evidence that a single peptide-MHC complex on a target cell can elicit a cytolytic T cell response. Immunity 4, 565-571 (1996).
Watanabe, K. et al. Target antigen density governs the efficacy of anti-CD20-CD28-CD3 ζ (chimeric antigen receptor-modified effector CD8+ T cells. J Immunol 194, 911-920 (2015).

1. An antibody or antigen binding fragment comprising any one or more of SEQ ID NOs: 4-9 or 22-27.

2. An antibody or antigen binding fragment comprising a heavy chain variable region having at least 80% sequence identity to SEQ ID NO:22-24.

3. The antibody or antigen binding fragment of claim 2, wherein the antibody or antigen binding fragment further comprises a light chain variable region having at least 80% sequence identity to SEQ ID NO:25-27.

4. An antibody or antigen binding fragment comprising a light chain variable region having at least 80% sequence identity to SEQ ID NO:25-27.

5. The antibody or antigen binding fragment of claim 4, wherein the antibody or antigen binding fragment comprises a heavy chain variable region having at least 80% sequence identity to SEQ ID NO:22-24.

6. An antibody or antigen binding fragment comprising a heavy chain variable region and a light chain variable region, wherein the heavy chain variable region comprises a HCDR1, HCDR2, and HCDR3 having at least 80%, 85%, 90%, 91%, 92%, 93%, 94%, 95%, 96%, 97%, 98%, or 99% sequence identity to SEQ ID NOs:28-30, respectively, and wherein the light chain variable region comprises a LCDR1, LCDR2, and LCDR3 having at least 80% sequence identity to SEQ ID NOs:31-33, respectively.

7. An antibody or antigen binding fragment comprising a heavy chain variable region and a light chain variable region, wherein the heavy chain variable region comprises a HCDR1, HCDR2, and HCDR3 of SEQ ID NOs:28-30, respectively, and wherein the light chain variable region comprises a LCDR1, LCDR2, and LCDR3 of SEQ ID NOs:31, 33 or “DVS”, respectively.

8. An antibody or antigen binding fragment comprising a heavy chain variable region and a light chain variable region, wherein the heavy chain variable region comprises a HCDR1, HCDR2, and HCDR3 having at least 80%, 85%, 90%, 91%, 92%, 93%, 94%, 95%, 96%, 97%, 98%, or 99% sequence identity to SEQ ID NOs:34-36, respectively, and wherein the light chain variable region comprises a LCDR1, LCDR2, and LCDR3 having at least 80% sequence identity to SEQ ID NOs:37, 39 or “DAS”, respectively.

9. An antibody or antigen binding fragment comprising a heavy chain variable region and a light chain variable region, wherein the heavy chain variable region comprises a HCDR1, HCDR2, and HCDR3 of SEQ ID NOs:34-36, respectively, and wherein the light chain variable region comprises a LCDR1, LCDR2, and LCDR3 of SEQ ID NOs:37, 39 or “DAS”, respectively.

10. An antibody or antigen binding fragment comprising a heavy chain variable region and a light chain variable region, wherein the heavy chain variable region comprises a HCDR1, HCDR2, and HCDR3 having at least 80%, 85%, 90%, 91%, 92%, 93%, 94%, 95%, 96%, 97%, 98%, or 99% sequence identity to SEQ ID NOs:40-42, respectively, and wherein the light chain variable region comprises a LCDR1, LCDR2, and LCDR3 having at least 80% sequence identity to SEQ ID NOs:43, 45 or “DDN”, respectively.

11. An antibody or antigen binding fragment comprising a heavy chain variable region and a light chain variable region, wherein the heavy chain variable region comprises a HCDR1, HCDR2, and HCDR3 of SEQ ID NOs:40-42, respectively, and wherein the light chain variable region comprises a LCDR1, LCDR2, and LCDR3 of SEQ ID NOs:43, 45 or “DDN”, respectively.

12. An antibody or antigen binding fragment comprising a heavy chain variable region and a light chain variable region, wherein the heavy chain variable region comprises sequence that is at least 80%, 85%, 90%, 91%, 92%, 93%, 94%, 95%, 96%, 97%, 98%, or 99% sequence identity to SEQ ID NO:22, and wherein the light chain variable region comprises sequence that is at least 80%, 85%, 90%, 91%, 92%, 93%, 94%, 95%, 96%, 97%, 98%, or 99% sequence identity to SEQ ID NO:25.

13. The antibody or antigen binding fragment of claim 12, wherein the heavy chain variable region comprises the sequence of SEQ ID NO:22.

14. The antibody or antigen binding fragment of claim 12 or 13, wherein the light chain variable region comprises the sequence of SEQ ID NO:25.

15. An antibody or antigen binding fragment comprising a heavy chain variable region and a light chain variable region, wherein the heavy chain variable region comprises sequence that is at least 80%, 85%, 90%, 91%, 92%, 93%, 94%, 95%, 96%, 97%, 98%, or 99% sequence identity to SEQ ID NO:23, and wherein the light chain variable region comprises sequence that is at least 80%, 85%, 90%, 91%, 92%, 93%, 94%, 95%, 96%, 97%, 98%, or 99% sequence identity to SEQ ID NO:26.

16. The antibody or antigen binding fragment of claim 12, wherein the heavy chain variable region comprises the sequence of SEQ ID NO:23.

17. The antibody or antigen binding fragment of claim 12 or 13, wherein the light chain variable region comprises the sequence of SEQ ID NO:26.

18. An antibody or antigen binding fragment comprising a heavy chain variable region and a light chain variable region, wherein the heavy chain variable region comprises sequence that is at least 80%, 85%, 90%, 91%, 92%, 93%, 94%, 95%, 96%, 97%, 98%, or 99% sequence identity to SEQ ID NO:24, and wherein the light chain variable region comprises sequence that is at least 80%, 85%, 90%, 91%, 92%, 93%, 94%, 95%, 96%, 97%, 98%, or 99% sequence identity to SEQ ID NO:27.

19. The antibody or antigen binding fragment of claim 12, wherein the heavy chain variable region comprises the sequence of SEQ ID NO:24.

20. The antibody or antigen binding fragment of claim 12 or 13, wherein the light chain variable region comprises the sequence of SEQ ID NO:27.

21. The antibody or antigen-binding fragment of any one of claims 1-20, wherein the antibody, or antigen binding fragment binds a TdT pepide with a K_{D} of about 10⁻³ M to about 10⁻⁹ M.

22. The antibody or antigen binding fragment of any one of claims 1-21, wherein the antibody is a human antibody, humanized antibody, recombinant antibody, chimeric antibody, an antibody derivative, neutralizing antibody, a veneered antibody, a diabody, a monoclonal antibody, a single domain antibody, or a single chain antibody.

23. The antigen binding fragment of any one of claims 1-22, wherein the antigen binding fragment is a single chain variable fragment (scFv), F(ab′)₂, Fab′, Fab, Fv, or rIgG.

24. The antibody or antigen binding fragment of any one of claims 1-23, further defined as being comprised in a chimeric polypeptide.

25. The antibody or antigen binding fragment of claim 24, wherein the chimeric polypeptide is a receptor or a multi-specific antibody.

26. A polypeptide comprising the antigen binding fragment of any one of claims 1-25.

27. The polypeptide of claim 26, wherein the polypeptide comprises at least two antigen binding fragments, wherein each antigen binding fragment is independently selected from an antigen binding fragment of any one of claims 1-25.

28. The polypeptide of claim 26 or 27, wherein the polypeptide is multivalent.

29. The polypeptide of any one of claims 26-28, wherein the polypeptide is bispecific.

30. The polypeptide of claim 29, wherein the polypeptide is a bi-specific antibody in which a first antigen binding domain of the antibody binds a TdT peptide and a second antigen binding domain of the antibody binds CD3.

31. A composition comprising the antibody or antigen binding fragment of any one of claims 1-25.

32. The composition of claim 31, wherein the composition comprises a chimeric receptor or a multi-specific antibody.

33. The composition of claim 32, wherein the chimeric receptor is a chimeric antigen receptor or a chimeric T-cell receptor (TCR) comprising TCR α and β chains.

34. The composition of claim 32, wherein the multi-specific antibody is a bi-specific antibody.

35. The composition of claim 32 or 34, wherein the composition is a bi-specific T-cell engager.

36. The composition of any one of claims 31-35, wherein the composition comprises a pharmaceutical excipient.

37. The composition of any one of claims 31-36, wherein the composition further comprises an adjuvant.

38. The composition of any one of claims 31-37, wherein the composition is formulated for administration parenterally, orthotopically, intradermally, subcutaneously, orally, transdermally, intramuscularly, intraperitoneally, intraperitoneally, intraorbitally, by implantation, by inhalation, intraventricularly, intranasally, intraarterially, intratracheally, intrapleurally, intratumorally, endoscopically, intralesionally, intracranially, percutaneously, regionally, systemically, by perfusion, in a tumor microenvironment, and/or by intravenous injection.

39. The composition of any one of claims 31-38, wherein the composition comprises at least two antibodies or antigen binding fragments.

40. One or more nucleic acids encoding the antibody or antigen binding fragment of any one of claims 1-25 or the polypeptide of any one of claims 26-f30.

41. A nucleic acid encoding a TdT peptide-specific antibody, wherein the nucleic acid has at least 70% sequence identity to one of SEQ ID NOS:4, 6, or 8.

42. A vector comprising the nucleic acid(s) of claim 41.

43. A host cell comprising the nucleic acid of claim 41 or the vector of claim 42.

44. The host cell of claim 43, wherein the host cell is a human cell, immune cell, immune effector cell, B cell, T cell, Chinese hamster ovary, NS0 murine myeloma cell, or PER.C6 cell.

45. A method of a making a cell comprising transferring the nucleic acid of claim 41 or the vector of claim 42 into a cell.

46. The method of claim 45, wherein the method further comprises culturing the cell under conditions that allow for expression of a polypeptide from the nucleic acid.

47. The method of claim 46, wherein the method further comprising isolating the expressed polypeptide.

48. The method of any one of claims 45-47, wherein the cell is a human cell, B cell, T cell, Chinese hamster ovary, NS0 murine myeloma cell, or PER.C6 cell.

49. A method for producing a polypeptide comprising transferring the nucleic acid of claim 41 or the vector of claim 42 into a cell and isolating polypeptides expressed from the nucleic acid.

50. The method of claim 49, wherein the cell is a human cell, B cell, T cell, Chinese hamster ovary, NS0 murine myeloma cell, or PER.C6 cell.

51. A method for treating or preventing cancer in a subject, the method comprising administering to the subject a therapeutically effective amount of the antibody or antigen binding fragment of any one of claims 1-25, the polypeptide of any one of claims 26-30, the composition of any one of claims 31-39, or the host cell of claim 43 or 44.

52. The method of claim 51, wherein the subject is a human subject.

53. The method of claim 51 or 52, wherein the cancer is hematological.

54. The method of claim 51 or 52, wherein the cancer is solid tumor.

55. The method of any one of claims 51-54, wherein the subject has one or more symptoms of cancer.

56. The method of claim 51-54, wherein the subject does not have any symptoms of cancer.

57. The method of any one of claims 51-56, wherein the subject has been diagnosed with cancer.

58. The method of any one of claims 51-56, wherein the subject has not been diagnosed with cancer.

59. The method of any one of claims 51-58, wherein the subject has not been previously vaccinated for cancer.

60. The method of any one of claims 51-59, wherein the antibody, antigen binding fragment, polypeptide, or cell is administered parenterally, orthotopically, intradermally, subcutaneously, orally, transdermally, intramuscularly, intraperitoneally, intraperitoneally, intraorbitally, by implantation, by inhalation, intraventricularly, intranasally, intraarterially, intratracheally, intrapleurally, intratumorally, endoscopically, intralesionally, intracranially, percutaneously, regionally, systemically, by perfusion, in a tumor microenvironment, and/or by intravenous injection.

61. The method of any one of claims 51-60, wherein the subject has been previously treated for cancer.

62. The method of any one of claims 51-61, wherein the subject is administered an additional cancer therapeutic or medical treatment.

63. The method of claim 62, wherein the additional cancer therapeutic or medical treatment comprises surgery, radiation, chemotherapy, drug therapy, hormone therapy, or a combination thereof.

64. A method for reducing the risk of cancer, preventing cancer, or delaying the onset of cancer in an individual, comprising administering a therapeutically effective amount to the subject the antibody or antigen binding fragment of any one of claims 1-25, the polypeptide of any one of claims 26-30, the composition of any one of claims 31-39, or the host cell of claim 43 or 44.

65. The method of claim 64, wherein the individual has one or more cancer risk factors.

66. The method of claim 64 of 65, wherein the individual is a smoker, is over 50 years of age, has a genetic marker, has a personal history, has a family history, has sun damage to the skin, or a combination thereof.

67. A method for evaluating a sample from a subject, the method comprising contacting a biological sample from the subject, or extract thereof, with at least one antibody, antigen binding fragment of any one of claims 1-25, the polypeptide of any one of claims 26-30, the composition of any one of claims 31-39, or the host cell of claim 43 or 44.

68. The method of claim 67, wherein the at least one antibody, antigen binding fragment, or polypeptide is operatively linked to a detectable label.

69. The method of claim 67 or 68, wherein the method further comprises incubating the antibody, antigen binding fragment, or polypeptide under conditions that allow for the binding of the antibody, antigen binding fragment, or polypeptide to antigens in the biological sample or extract thereof.

70. The method of any one of claims 67-69, wherein the method further comprises detecting the binding of an antigen to the antibody, antigen binding fragment, or polypeptide.

71. The method of any one of claims 67-70, wherein the method further comprises contacting the biological sample with at least one capture antibody, antigen, or polypeptide.

72. The method of claim 71, wherein the at least one capture antibody, antigen binding fragment, or polypeptide comprises at least one antibody of claims 1-15.

73. The method of claim 71 or 72, wherein the capture antibody is linked to a solid support.

74. The method of any one of claims 67-73, wherein the biological sample comprises a a blood sample, urine sample, fecal sample, nasopharyngeal sample, cerebrospinal fluid sample, cheek scraping sample, nipple aspirate sample, biopsy sample, or a combination thereof.

75. The method of any one of claims 67-74, wherein the individual has a higher risk of cancer than the general population.

76. A method for diagnosing cancer in a subject, the method comprising contacting a biological sample from the subject, or extract thereof, with at least one antibody, antigen binding fragment of any one of claims 1-25 or with the polypeptide of any one of claims 26-30.

77. The method of claim 76, wherein the at least one antibody, antigen binding fragment, or polypeptide is operatively linked to a detectable label.

78. The method of claim 76 or 77, wherein the method further comprises incubating the antibody, antigen binding fragment, or polypeptide under conditions that allow for the binding of the antibody, antigen binding fragment, or polypeptide to antigens in the biological sample or extract thereof.

79. The method of any one of claims 76-78, wherein the method further comprises detecting the binding of an antigen to the antibody, antigen binding fragment, or polypeptide.

80. The method of any one of claims 76-79, wherein the method further comprises contacting the biological sample with at least one capture antibody, antigen, or polypeptide.

81. The method of claim 80, wherein the at least one capture antibody, antigen, or polypeptide comprises at least one antibody, antigen, or polypeptide of claims 1-15.

82. The method of claim 80 or 81, wherein the capture antibody is linked to a solid support.

83. The method of any one of claims 76-82, wherein the biological sample comprises a a blood sample, urine sample, fecal sample, nasopharyngeal sample, cerebrospinal fluid sample, cheek scraping sample, nipple aspirate sample, biopsy sample, or a combination thereof.

84. A method of treating an individual for cancer, comprising the step of providing a therapeutically effective amount of the antibody or antigen binding fragment of any one of claims 1-25, the polypeptide of any one of claims 26-30, the composition of any one of claims 31-39, or the host cell of claim 43 or 44 to an individual measured as having cancer cells expressing a TdT peptide, said measuring using the antibody or antigen binding fragment of any one of claims 1-25, the polypeptide of any one of claims 26-30, the composition of any one of claims 31-39, or the host cell of claim 43 or 44.
